(19) United States
(12) Patent Application Publication
(10) Pub. No.: US 2013/0268029 A1
(43) Pub. Date: Oct. 10, 2013

(54) Implantable Transponder Systems and Methods

(71) Applicant: Micro Transponder, Inc., Austin, TX (US)

(72) Inventors: Lawrence Cauller, Plano, TX (US); Richard Weiner, Dallas, TX (US)

(21) Appl. No.: 13/908,592

(22) Filed: Jun. 3, 2013

Related U.S. Application Data
(63) Continuation of application No. 12/323,854, filed on Nov. 26, 2008, now Pat. No. 8,457,757.
(60) Provisional application No. 60/990,278, filed on Nov. 26, 2007.

(51) Int. Cl. A61N 1/372

(52) U.S. Cl. CPC A61N 1/37229
USPC 607/61

(57) ABSTRACT

A method and system for providing electrical stimulation to tissue includes implanting one or more battery-free micro-transponders having spiral antennas into tissue. Energy is provided wirelessly to the plurality of micro-transponders. Tissue is stimulated using the energy.

### CROSS-REFERENCE TO RELATED APPLICATIONS

The present application is a continuation application of U.S. application Ser. No. 12/323,854 filed Nov. 26, 2008, which claims priority to U.S. Provisional Patent Application No. 60/990,278 filed on Nov. 26, 2007, both of which are incorporated herein by reference as if reproduced in their entirety.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

The present application relates to implantable peripheral nerve stimulation and sensor systems and more particularly to implantable micro-transponders with identified reply.

### SUMMARY

In one embodiment the disclosure includes a linear implantable device array comprising a plurality of implantable devices, wherein each of the plurality of implantable devices comprises an antenna, an energy storage circuit connected to the antenna, a stimulator circuit connected to the energy storage circuit and applying pulse to a plurality of electrodes, and a connecting material, wherein the connecting material connects each of the implantable devices to form a physically-connected array of implantable devices, wherein the implantable devices are collectively housed in a cannula, and wherein the cannula is configured to implant the physically-connected array of implantable devices into a tissue at a single injection point.

In another embodiment the disclosure includes implanting into tissue an array of independent implantable devices, each independent implantable device including an antenna and electrodes, wherein each of the independent implantable devices is physically connected to at least another one of the independent implantable devices in the array with a connecting material, and wherein the physically-connected array of independent implantable devices is housed within a cannula prior to being implanted into the tissue, providing energy through skin to each of the antennas at the same time using a single control device, communicating instructions from the single control device to each of the independent implantable devices using the energy, receiving response replies from each of the independent implantable devices by the single control device, and stimulating the tissue using one or more of the independent implantable devices in accordance with the instructions.

These and other features will be more clearly understood from the following detailed description taken in conjunction with the accompanying drawings and claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a block diagram depicting a reply micro-transponder in accordance with an embodiment.

FIG. 2 is a block diagram depicting an identification reply micro-transponder in accordance with an embodiment.

FIG. 3 is a block diagram depicting a data reply micro-transponder in accordance with an embodiment.

FIG. 4 is a circuit diagram depicting an asynchronous stimulation micro-transponder in accordance with an embodiment.

FIG. 5 includes graphs summarizing the variance of stimulus frequency, current amplitudes and stimulus pulse duration in accordance with an embodiment.

FIG. 6 is a circuit diagram of an external trigger micro-transponder in accordance with an embodiment.

FIG. 7 is a chart of the demodulation of an external interrupt trigger signal by differential filtering in accordance with an embodiment.

FIG. 8 includes graphs summarizing of micro-transponder operation in accordance with an embodiment.

FIG. 9 is a circuit diagram of a micro-transponder in accordance with an embodiment.

FIG. 10 is an illustration of a laminar spiral micro-foil in accordance with an embodiment.

FIG. 11 is an illustration of a gold laminar spiral micro-foil in accordance with an embodiment.

FIG. 12 is a circuit diagram depicting a depolarizing micro-transponder driver circuit, in accordance with an embodiment.

FIG. 13 is a graph depicting a stimulus voltage in accordance with an embodiment.

FIG. 14 is a block diagram depicting a micro-transponder system, in accordance with an embodiment.

FIG. 15 is a circuit diagram depicting a driver circuit, in accordance with an embodiment.

FIG. 16 is a circuit diagram depicting a driver circuit, in accordance with an embodiment.

FIG. 17 is a circuit diagram depicting a driver circuit, in accordance with an embodiment.

FIG. 18 is a circuit diagram depicting a driver circuit, in accordance with an embodiment.

FIG. 19A is an illustration of a deployment of a plurality of wireless micro-transponders distributed throughout subcutaneous vascular beds and terminal nerve fields consistent with the present innovations.

FIG. 19B is an illustration of a deployment of wireless micro-transponders to enable coupling with deep micro-transponder implants consistent with the present innovations.

FIG. 19C is an illustration of a deployment of wireless micro-transponders to enable coupling with deep neural micro-transponder implants consistent with the present innovations.

FIG. 20 shows an expanded view of an example of a micro-transponder bio-delivery system.

FIG. 21 is an illustration of a fabrication sequence for spiral type wireless micro-transponders consistent with the present innovations.

FIG. 22 shows an example of loading a hypodermic cannula with micro-transponder array during manufacturing process.

FIG. 23 shows an example of a micro-transponder ejection system.

FIG. 24(a) shows a cross-sectional view of an example of micro-transponder implantation process.

FIG. 24(b) shows a cross-sectional view of a micro-transponder ejection system immediately after an implantation process.

FIG. 25 shows an example of a micro-transponder ejection system immediately after ejection.

FIG. 26 shows an example of a micro-transponder array.

FIG. 27(a) shows a side view of the micro-transponder array of FIG. 26.

FIG. 27(b) shows a plan view of the micro-transponder array of FIG. 26.

FIG. 28 shows another example of a micro-transponder array.

FIG. 29(a) shows a side view of the micro-transponder array of FIG. 28.

FIG. 29(b) shows a plan view of the micro-transponder array of FIG. 28.

FIG. 30 shows a sectional view of another embodiment of a micro-transponder array.

FIG. 31 is a block diagram showing an addressable transponder system, in accordance with an embodiment.

FIG. 32 is a block diagram showing an addressable transponder system, in accordance with an embodiment.

FIG. 33 is a block diagram showing an addressable transponder system, in accordance with an embodiment.

FIG. 34 is a block diagram showing an addressable transponder system, in accordance with an embodiment.

FIG. 35 is a circuit diagram depicting a tissue model.

FIG. 36 is a wireless implant platform, in accordance with an embodiment.

FIG. 37 is a wireless implant platform, in accordance with an embodiment.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

A variety of medical conditions involve disorders of the neurological system within the human body. Such conditions may include paralysis due to spinal cord injury, cerebral palsy, polio, sensory loss, sleep apnea, acute pain, and so forth. One characterizing feature of these disorders may be, for example, the inability of the brain to neurologically communicate with neurological systems dispersed throughout the body. This may be due to physical disconnections within the neurological system of the body, and/or to chemical imbalances that can alter the ability of the neurological system to receive and transmit electrical signals, such as those propagating between neurons.

Advances in the medical field have produced techniques aimed at restoring or rehabilitating neurological deficiencies leading to some of the above-mentioned conditions. However, such techniques are typically aimed at treating the central nervous system and therefore are quite invasive. These techniques include, for example, implanting devices, such as electrodes, into the brain and physically connecting those devices via wires to external system adapted to send and receive signals to and from the implanted devices. While beneficial, the incorporation of foreign matter into the human body usually presents various physiological complications, including surgical wounds and infection, rendering these techniques very challenging to implement.

The present application discloses new approaches to methods and systems for providing electrical stimulation to tissue includes implanting one or more battery-free micro-transponders having spiral antennas into tissue. Energy is provided wirelessly to the plurality of micro-transponders. Tissue is stimulated using the energy.

The disclosed innovations, in various embodiments, provide one or more of at least the following advantages. However, not all of these advantages result from every one of the innovations disclosed, and this list of advantages does not limit the various claimed inventions.
External power source and control
Minimally invasive stimulation units

The unprecedented miniaturization minimally invasive biomedical implants made possible with this wireless micro-transponder technology would enable novel forms of distributed stimulation or high resolution sensing using micro-implants so small that implantation densities of 100 per square inch of skin are feasible. The micro-transponders may operate without implanted batteries. Micro-transponders communicate information and may be powered without wire connections. Additionally, micro-transponders may be powered without wire connections that pass through the patient's skin or organ layers. The micro-transponders may receive energy and information and may transmit energy and information using the flux of an electromagnetic fields between internal inductance coils within the micro-transponders and external inductance coils placed above the surface of the overlying skin.

Power and modulated signals may be communicated wirelessly using the near-field magnetic coupling between two coils of conductive material. The coils of conductive material exhibit an inductance which in conjunction with a capacitance forms an LC resonator that may be tuned to resonate at specific frequencies. Two coils will communicate most efficiently when they are tuned to the same or related frequencies. Harmonic relationships between specified frequencies make it possible for different, harmonically related, frequencies to transfer power effectively, allowing coils of significantly different size to communicate with a suitable efficiency.

Recognizing this relationship between frequencies, references to tuning a pair of coils to the “same frequency” may include tuning the pair of coils to harmonically related frequencies. By energizing a first coil at a given frequency, an electromagnetic field is generated. By placing a second coil in the electromagnetic field, current is generated in the second coil. When the resonant frequencies of the coils are the same or of a harmonically related frequency, the generated current is maximized. Generated current may be typically stored in a capacitor and may be used to energize system elements.

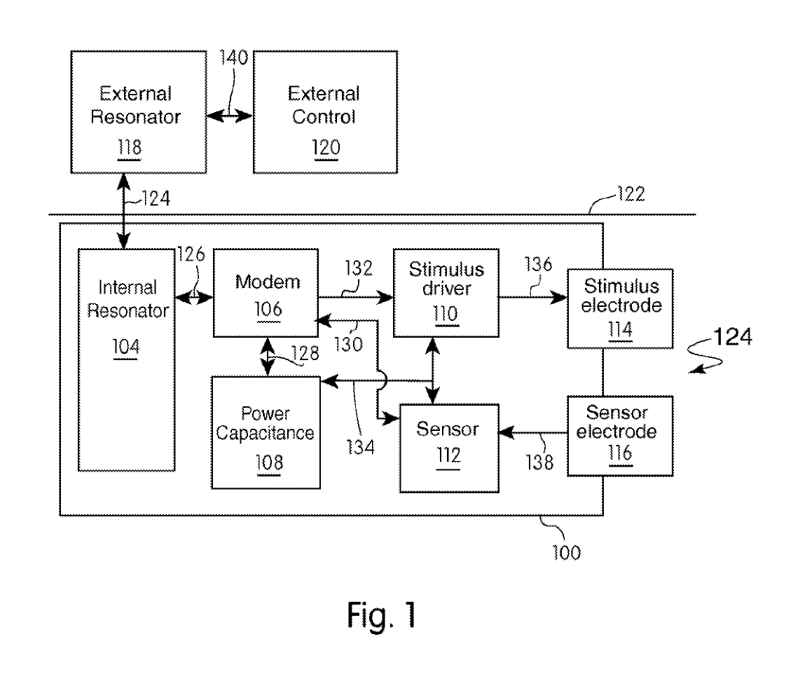

With reference to FIG. 1, a block diagram depicts a micro-transponder 100 in accordance with an embodiment. The micro-transponder 100 may be implanted in tissue 124 beneath a layer of skin 122. The micro-transponder 100 may be used to sense neural activity in the tissue 124 and communicate data to an external control 120 in response. The micro-transponder 100 may be used to provide electrical stimulation to the tissue 124 in response to a signal from an external control 120. The electrodes 114 and 116 may be designed to enhance the electrical interface between the electrodes 114 and 116 and neurons of peripheral nerves.

The micro-transponder 100 may wirelessly interact with other systems. The micro-transponder 100 may interact via direct electrical connection with other systems. Typically, the micro-transponder 100 interacts wirelessly with an external control system 120 including an external resonator 118. The micro-transponder 100 may communicate via a direct electrical connection with other micro-transponders (not shown) implanted within the body.

The micro-transponder 100 enables delivery of electrical signals to peripheral nerves. These signals may be configured to stimulate peripheral nerves distributed throughout subcutaneous tissue 124. The micro-transponder 100 enables the detection of electrical signals in peripheral nerves. The detected electrical signals may be indicative of neural spike signals.

Micro-transponder 100 includes an internal resonator 104. The internal resonator 104 might be connected to a modulator-demodulator 106, to modulate information onto outgoing signals and/or retrieve information from incoming signals. The modulator-demodulator 106 may modulate or demodulate identification signals. The modulator-demodulator 106 may demodulate trigger signals. The modulator-demodulator 106 may receive signals from an impulse sensor 112. The modulator-demodulator 106 may provide trigger signals or other data to a stimulus driver 110. The impulse sensor 112 may be connected to a sensor electrode 116. The impulse sensor 112 may generate a signal when a current is detected at the sensor electrode. 116. The stimulus driver 110 may be connected to stimulus electrodes 114. The stimulus driver 110 typically generates a stimulation voltage between the stimulus electrodes 114 when a trigger signal is received.

The internal resonator 104 provides energy to a power storage capacitance 108, which stores power received by the internal resonator 104. The power capacitance 108 may provide power 134 to the other components, including the stimulus driver 110, the impulse sensor 112 and the modem 106.

In operation, an external control 120, typically a computer or other programmed signal source, may provide commands 140 regarding sensing or stimulation for the micro-transponder 100. The commands 140 are provided to an external resonator 118 and may initiate stimulation cycles, poll the devices, or otherwise interact with the micro-transponder 100. The external resonator 118 is tuned to resonate at the same frequency, or a related frequency, as the internal resonator 104. Signal 126 are generated by the external resonator 118, resonated at the tuned frequency. The signal 126 may be a power signal without any modulated data. The signal 126 may be a power signal including modulated data, where the modulated data typically reflects commands 140 provided by the external control 120 such as identification information or addresses. It should be recognized that a power signal without modulated data may communicate timing data, such as a trigger signal, in the presentation or timing of the power signal.

The internal resonator 104 receives signals 126 from the external resonator 118. The internal resonator 104 provides a received signal 126 to the modulator-demodulator (modem) 106. The modem 106 may demodulate instructions 132 from the received signal. Demodulated instructions 132 may be provided to the stimulus driver 110. The modem 106 may pass the power signal 128 to the power capacitance 108. The power capacitance 108 may store the power signal 128. The power capacitance 108 may provide power to the stimulus driver 110. The power capacitance 108 may provide power to the impulse sensor 112. The stimulus driver 110 may provide a stimulus signal 136 to the stimulus electrode 114. The stimulus driver 110 may provide a stimulus signal 136 to the stimulus electrode 114 in response to an instruction 132. The stimulus driver 110 may provide a stimulus signal 136 to the stimulus electrode 114 in response to a power signal 134.

The modem 106 may provide an instruction 130 to impulse sensor 112. When an impulse is sensed in the tissue 124, the sensor electrode sends an impulse signal 138 to impulse sensor 112. The impulse sensor 112 sends a sensed impulse signal 130 to the modem 106. In response to the sensed impulse signal 112, the modem 116 may modulate an identification signal 126 onto a power signal 128. The internal resonator 104 generates a communication signal 124 including a modulated identification signal 126. The external resonator 118 receives the communication signal 124. Data 140 is provided to the external control 120.

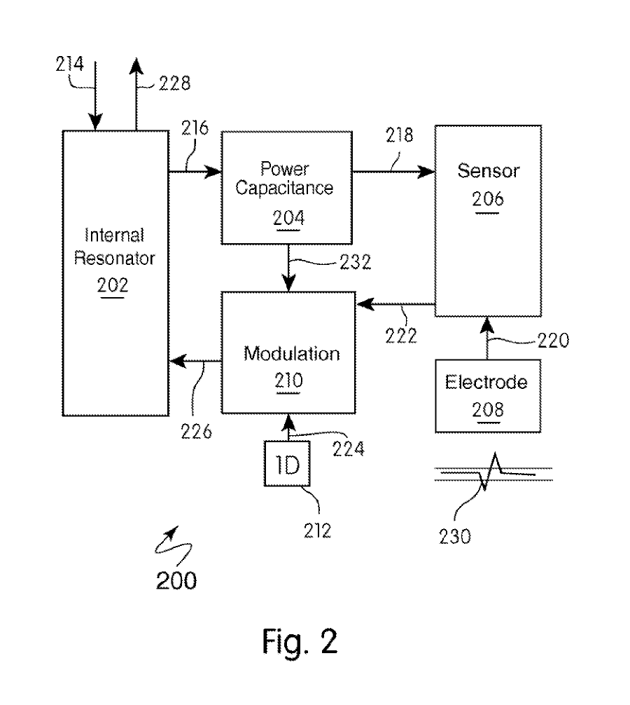

With reference to FIG. 2, a block diagram depicts a sensing micro-transponder 200, in accordance with an embodiment. An internal resonator 202 receives an operation signal 214, where the operation signal 214 has been transmitted inductively by an external resonator (not shown). The operation signal 214 may include instructions, commands, address data or any other suitable data. The internal resonator 202 provides a power signal 216 to a power capacitance 204. The power capacitance 204 may subsequently provide power 218 to an impulse sensor 206, a modem 210, or any appropriate electrical component. The impulse sensor 206 is connected to a sensor electrode 208 placed proximate to peripheral nerve tissue 230. When an impulse passes through the peripheral nerve tissue 230, a charge is generated on the sensor electrode 208. The sensor electrode 208 provides a signal 220 to the impulse sensor 206. The impulse sensor 206 provides a signal to an identification modulator 210. The identification modulator 210 receives a power signal 232 from the power capacitance 204. The identification modulator 210 generates a modulated identification signal 226 using identification data 212. The internal resonator 202 generates a communication signal 228. An external resonator (not shown) receives the communication signal 228.

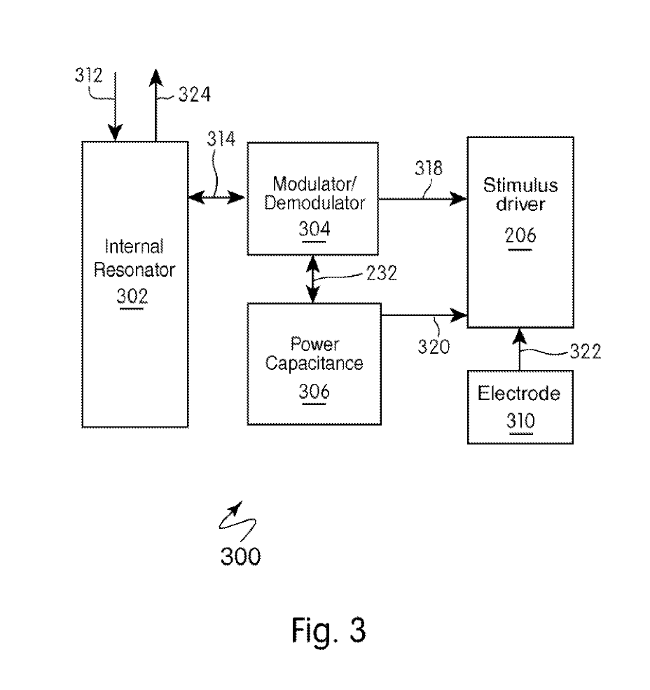

With reference to FIG. 3, a block diagram depicts a micro-transponder 300 including data reply in accordance with an embodiment. An internal resonator 302 receives an operation signal 312 from an external resonator (not shown). The operation signal 312 may include data, such as identification information, addressing, commands, instruction or other suitable data. The internal resonator 302 provides a received signal to a modem 304. The internal resonator 302 provides a power signal 316 to a power capacitance 306. The modem 304 demodulates data 318 that has been modulated on the received signal 314. The data 318, typically a trigger signal, is provided to the stimulus driver 308. The stimulus driver 308 receives a power signal from a power capacitance 306. The stimulus driver 308 provides stimulation energy 322 to a stimulation electrode 310 in response to receiving the trigger signal 318. The modem 304 receives power 316 from the power capacitance 306. Modem 304 generates a data reply signal 314 in response to data 318. The internal resonator 302 generates a communication signal 324. An external resonator (not shown) receives the communication signal 324.

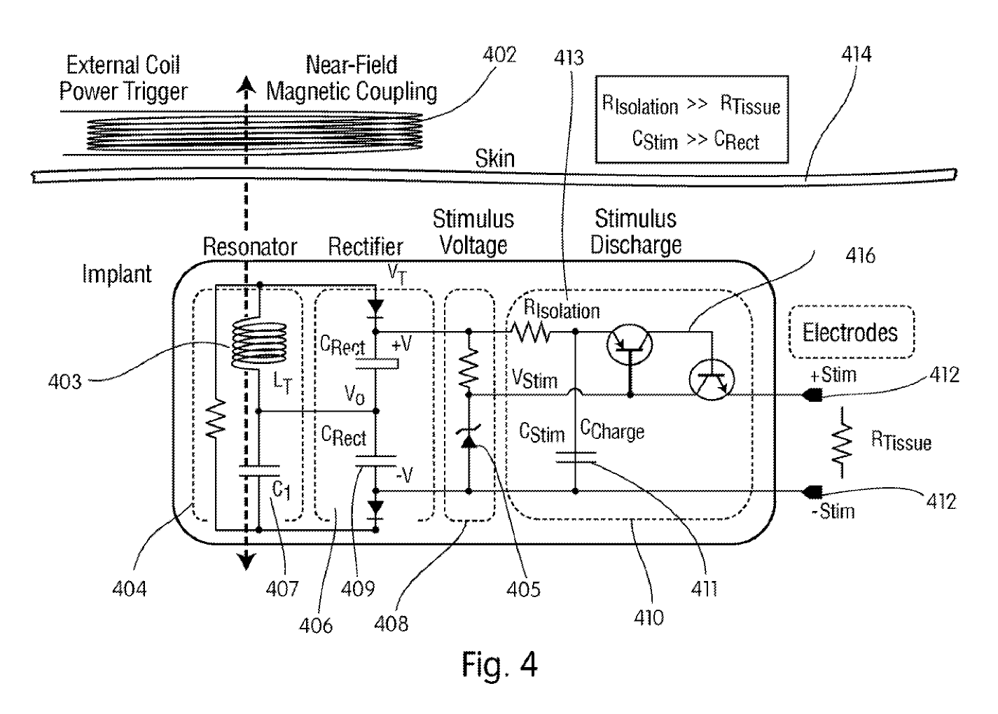

With reference to FIG. 4, a circuit diagram depicts a wireless micro-transponder having independent auto-triggering operation, in accordance with one embodiment. As shown by the circuit diagram, the auto-triggering micro-transponder includes a resonator element 404, a rectifier element 406, a stimulus voltage element 408, a stimulus discharger element 410, and one or more electrodes 412. The resonator element 404 includes a coil (LT) component 403 that is coupled to a capacitor (CT) component 407. The resonator element 404 is configured to oscillate at a precise frequency that depends upon the values of the coil component 403 and capacitor component 407.

The resonator element 404 is coupled to the rectifier element 406 which is in turn coupled to the stimulus voltage element 408 and the stimulus discharger element 410. The rectifier element 406 and the stimulus voltage element 408 are both coupled in parallel to capacitors 409. In addition, the stimulus discharger element 410 is coupled to electrodes 412, thereby electrically connecting the stimulus discharger element 410 to neural conduction tissue, such as axons. It should be appreciated that in certain embodiments, a voltage booster component may be inserted immediately after, to the rectifier element 406 to boost the supply voltage available for stimulation and operation of the integrated electronics beyond the limits generated by the miniaturized LC resonant tank circuit. This voltage booster can enable electro stimulation and other micro-transponder operations using the smallest possible LC components which may generate relatively small voltages (<0.5V). Examples of high efficiency voltage boosters include charge pumps and switching boosters using low-threshold Schottky diodes. However, it should be understood that any appropriate conventional high efficiency voltage booster may be utilized in this capacity.

In this circuit configuration, the auto-triggering micro-transponder 400 can employ a bistable silicon switch 416 to oscillate between the charging phase that builds up a charge (Vcharge) on the stimulus capacitor 411 and the discharge phase that can be triggered when the charge (Vcharge) reaches the desired stimulation voltage (Vstim). The discharge phase begins with closing the switch 418 and discharging the capacitor through the stimulus electrodes 412. A single resistor 413 is used to regulate the stimulus frequency by limiting the charging rate of the stimulus capacitor 411. The breakdown voltage of a zener diode 405 is configured to set the desired stimulus voltage (Vstim). When Vcharge is equal to Vstim, the switch 416 closes, closing switch 418 and discharging the capacitor 411 into the electrodes 412. The electrodes 412 may be formed of gold, a platinum iridium alloy or any other suitable material. Switches 416 and 418 may typically be bipolar devices, field-effect transistors, or any other suitable device.

The stimulus peak amplitude and duration are largely determined by the effective tissue resistance, independent of the applied power intensity. Effective tissue resistance may vary depending on the type of tissue being stimulated, for example, skin, muscle, fat, etc. However, increasing the power may increase the stimulation frequency by reducing the time required to charge the stimulation capacitor 411 to the stimulus voltage Vstim.

The auto-triggering micro-transponder 400 operates without timing signals from the power source 402 and auto-triggers repetitive stimulation independently. As a result, the stimulation generated by a plurality of such auto-triggering micro-transponders 400 would be asynchronous in phase and somewhat variable in frequency from one stimulator to another depending upon the effective transponder voltage induced by each transponder. Such asynchronous stimulation may evoke the sort of disordered pins and needles or tingling sensations of parasthesias that are associated with stimulation methods that most effectively block pain signals.

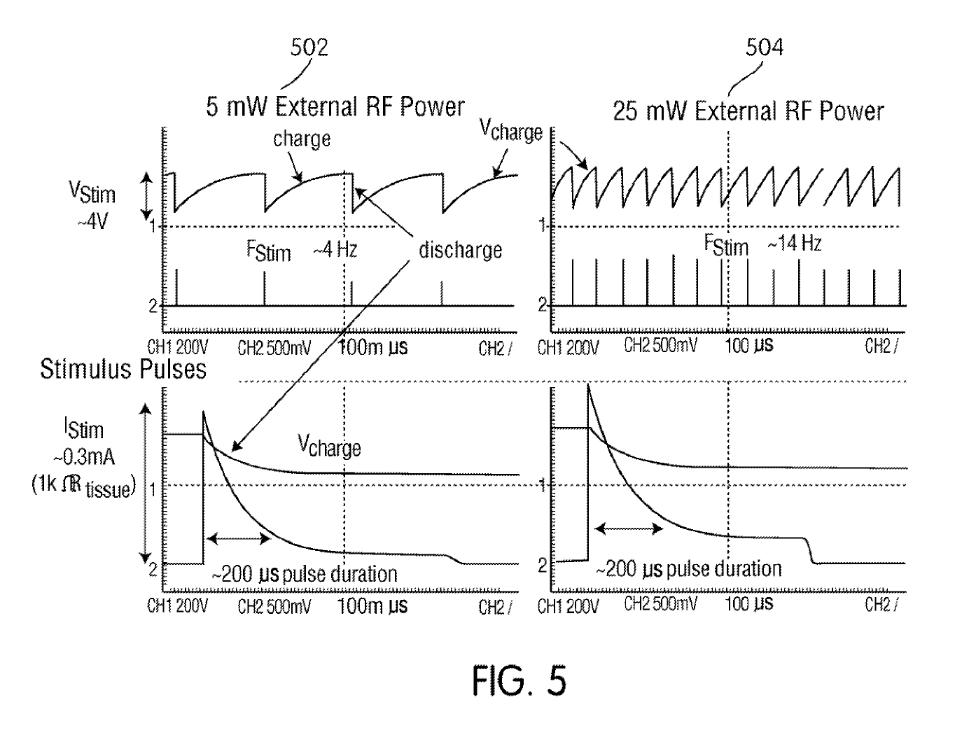

FIG. 5 presents several graphs that illustrate variations of wireless micro-transponder stimulus frequencies stimulus current peak amplitudes and stimulus pulse durations vary under different device settings and external RF power input conditions, in accordance with an embodiment.

In the first graph 502, the external RF power input is set at 5 milliwatts (mW) resulting in a stimulus frequency of 4 Hertz (Hz). As discussed previously, the stimulus frequency is a function of transmitted power as the received power directly affects the time it takes to charge a stimulus capacitor to the stimulus voltage (Vstim). This direct relationship between RF power and stimulus frequency is clearly shown in graph 502, where the external RF power is ramped up to 25 mW, which results in a significant increase in stimulus frequency to 14 Hz. It should be understood, however, that these are just examples of the effect of RF power input settings on stimulus frequency. In practice, the effects of the RF power input setting on stimulus frequency may be magnified or diminished depending on the particular application, for example, depth of implantation, proximity to interfering body structures such as bones, organs, etc. and device settings.

While RF intensity controls stimulus frequency, the stimulus voltage (Vstim) is typically controlled by the transponder zener diode element. The effect of stimulus voltage upon the stimulus current peak amplitude and pulse duration is further determined by the resistive properties of the tissue surrounding the micro-transponder.

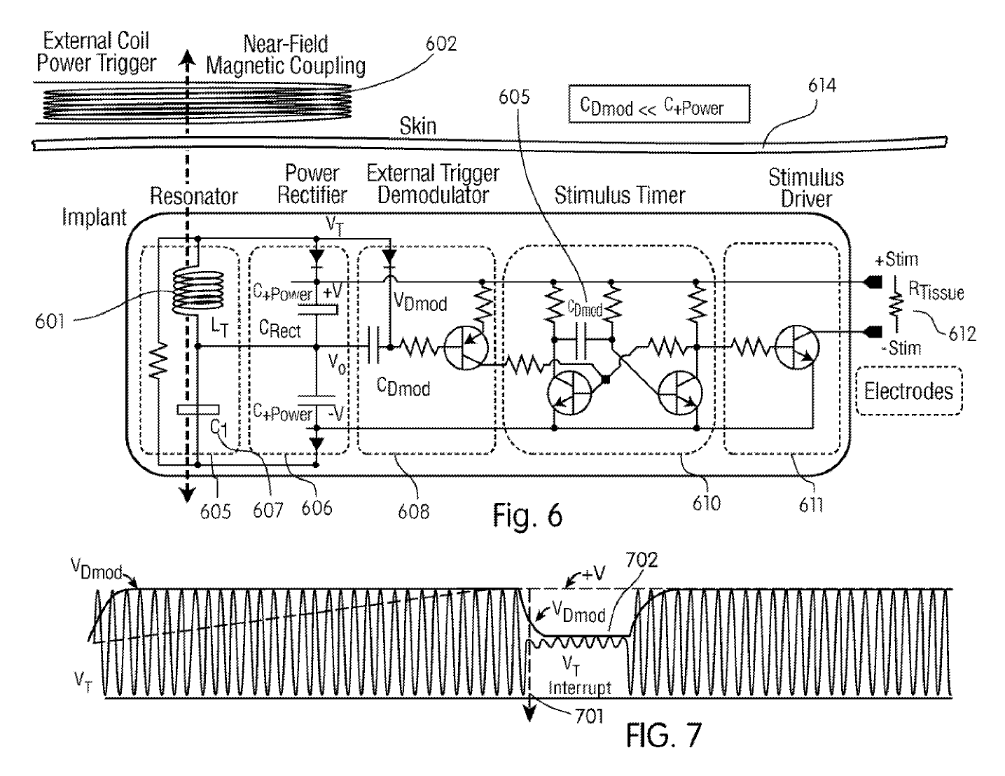

FIG. 6 is an illustration of a circuit diagram for a wireless micro-transponder 600 with an external trigger signal demodulator element 608 to synchronize the stimuli delivered with a plurality of other wireless micro-transponders, in accordance with an embodiment. As depicted, herein, the wireless transponder design of FIG. 5 is modified to include an external trigger signal demodulator element 608 so that the stimulus discharge can be synchronized by a trigger signal from an external RF power field.

The modified circuit includes a resonator element 604, a rectifier element 606, an external trigger demodulator element 608, a stimulus timer element 610, a stimulus driver element 611, and one or more electrodes 612. The resonator element 604 includes a coil component (LT) 601 that is coupled to a capacitor component (CT) 607. The resonator element 604 is configured to oscillate at a determined frequency depending on the value of the LC components LT 601 and CT 607.

The resonator element 604 is coupled to a rectifier element 606, which is in turn coupled to the external trigger demodulator element 608, the stimulus timer element 610 and the stimulus driver element 611. The rectifier element 607 and the stimulus timer element 608 are both coupled in parallel to power capacitors (Cpower) 609. In addition, the stimulus driver element 611 is coupled to electrodes 612, typically formed of gold or a platinum iridium alloy, thereby electrically connecting the stimulus driver element 611 to neural conduction tissue, such as axons.

It should be appreciated that, in certain embodiments, a standard voltage booster component (not shown) can be inserted immediately after the rectifier element 606 to boost the supply voltage available for stimulation and operation of integrated electronics beyond the limits generated by the miniaturized LC resonant tank circuit. A voltage booster may enable electro-stimulation and other micro-transponder operations using the smallest possible LC components, which may generate relatively small voltages, for example, less than 0.5 Volts. Examples of typical high efficiency voltage boosters include charge pumps and switching boosters using low-threshold Schottky diodes. However, it should be understood that any suitable type of conventional high efficiency voltage booster may be utilized in this capacity.

As show in FIG. 7, the external synchronization-trigger circuit configuration of FIG. 6 may employ a differential filtering method to separate the trigger signal, consisting of a sudden power interruption 701, from the slower drop in transponder power voltage 702 during the interruption. In particular, the circuit configuration of FIG. 6 may utilize a separate capacitor (CDur) 605 in the stimulus timer element 610, to set the stimulus duration using a mono-stable multi-vibrator. Stimulus intensity can be controlled externally by the intensity of the applied RF power field generated by the external FR power coil 602. As the RF power field is modulated, the timing and frequency of stimuli from each of the micro-transponders under the RF power coil 602 are synchronized externally.

Using the external synchronization-trigger circuit configuration of FIG. 6, the degree of spatio-temporal control of complex stimulus patterns is essentially unlimited. In certain embodiments, the circuit configuration of the external synchronization-trigger circuit can be further modified so that it is configured to demodulate the unique identity code of each micro-transponder. This essentially permits the independent control of each micro-transponder via RF signals. This added capability can provide a method to mediate the spatio-temporal dynamics necessary to restore natural sensations with artificial limbs or enable new sensory modalities, for example feeling infrared images, etc.

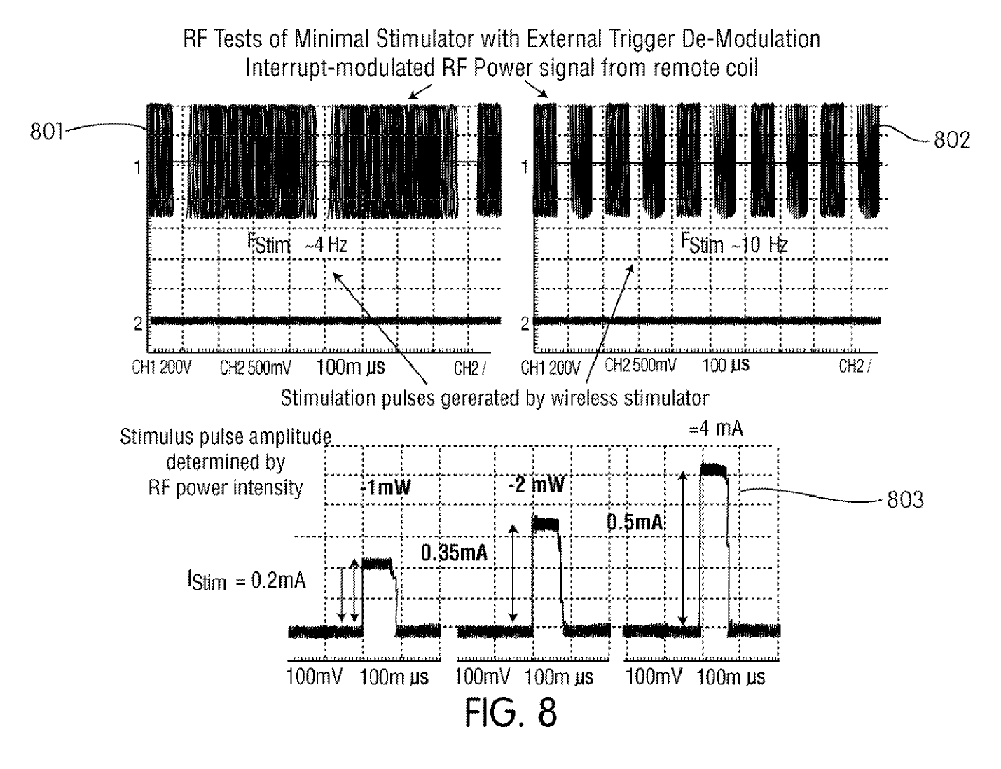

FIG. 8 presents several graphs that summarize the results from tests of a wireless micro-transponder (with an external interrupt trigger de-modulator element) under different device settings and external RF power input conditions, in accordance with one embodiment. In the first graph 801, the external RF power coil modulates the RF power field to communicate a first trigger signal setting, which results in a stimulus frequency of 2 Hz. As discussed previously, the stimulus frequency is controlled by a trigger signal created when the RF power coil modulates the RF power field. The stimulus frequency is therefore directly related to the RF power field modulation frequency as shown in the second graph 802, where the stimulus frequency equals 10 Hz.

Whereas the stimulus frequency is controlled by external RF power field modulation settings, the stimulus current peak amplitude is controlled by the RF power intensity setting, as shown in the third graph 803. That is, the stimulus current peak amplitude is directly related to the RF power intensity setting. For example, an RF power intensity setting of 1 mW produces a stimulus current peak amplitude of 0.2 milliamps (mA), a RF power intensity setting of 2 mW produces a stimulus current peak amplitude of 0.35 mA, and a RF power intensity setting of 4 mW produces a stimulus current peak amplitude of 0.5 mA. It should be understood, however, that these are just examples of how RF power intensity setting affects stimulus current peak amplitude. In practice, the effects of the RF power intensity setting on stimulus current peak amplitude may be magnified or diminished depending on the particular application (e.g., depth of implantation, proximity to interfering body structures such as bone, etc.) and device settings.

Whereas the stimulus frequency is controlled by an external RF power field modulation settings, the stimulus current peak amplitude is controlled by the RF power intensity setting as shown in the third graph 803. That is, the stimulus current peak amplitude is directly related to the RF power intensity setting. For examples, an RF power intensity setting of 1 mW produces a stimulus current peak amplitude of 0.2 mA, an RF power intensity setting of 2 mW produces a stimulus current peak amplitude of 0.35 mA, and an RF power intensity setting of 4 mW produces a stimulus current peak amplitude of 0.5 mA. It should be understood, however, that these are just examples of how RF power intensity settings affect stimulus current peak amplitude. In practice, the effects of the RF power intensity setting on stimulus current peak amplitude may be magnified or diminished depending on the particular application, for example, the depth of implantation, proximity to interfering body structures such as bones, etc., and device settings.

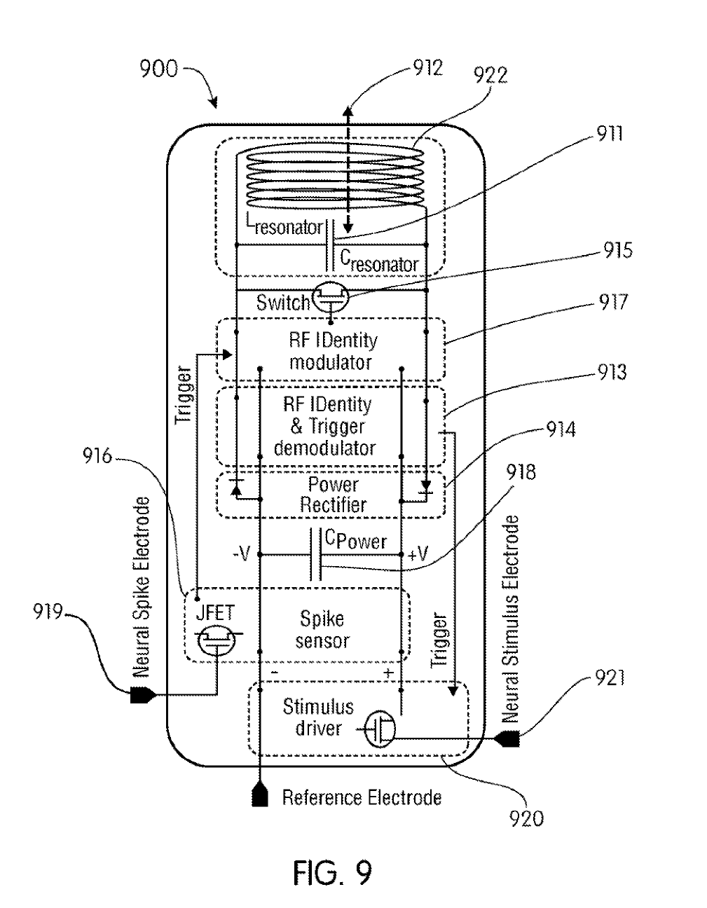

With reference to FIG. 9, a block diagram depicts a micro-transponder 900 in accordance with an embodiment. The micro-transponder 900 includes electrical components adapted to electrically interface with neurons of peripheral nerves. The micro-transponder 900 includes electrical components that enable the micro-transponder 900 to wirelessly interact with systems external to the micro-transponder 900. These systems may include other transponders implanted within the body. These systems may include external coils. These systems may include a receiver.

The wireless capability of the micro-transponder 900 enables the delivery of electrical signals to the peripheral nerve tissue. The wireless capability of the micro-transponder 900 enables communication in response to sensed signals in the peripheral nerve tissue. These may include signals indicative of neural spike signals. These may include signals configured to stimulate peripheral nerves distributed throughout the subcutaneous tissue.

The micro-transponder 900 includes coils 922 coiled about a central axis 912. The coil 922 is coupled in parallel to a capacitor 911 and to an RF identity modulator 917 via switch 915. The RF identity modulator 917 is coupled to an RF identity and trigger demodulator 913, which in turn is coupled to a rectifier 914. The rectifier 914 and the spike sensor 916 are both coupled in parallel to a capacitor 918. In addition, the spike sensor 916 is coupled to a neural spike electrode 919, thereby electrically connecting the spike sensor 916 to neural transmission tissue, such as neurons. Similarly, the neural stimulus electrode 921 also connects the stimulus driver 920 to neural conduction tissue such as axons.

The spike sensor 916 is made up of one or more junction field effect transistors (JFET). As will be appreciated by those skilled in the art, the JFET may include MOSFETS or any other suitable device. The sensors, drivers and other electronic components described in the present application may be fabricated using standard small scale or very large scale integration (VLSI) methods.

Further, the spike sensor 916 is coupled to the RF identity modulator 917, which is adapted to modulate an incoming/carrier RF signal in response to neural spike signal detected by the spike sensor 916. In an embodiment, the neural electrodes such as the neural spike electrode 919 and the neural stimulus electrode 921 to which the spike sensor 916 and the stimulus driver 920 are connected, respectively, may be bundled and configured to interface with neural conduction (axon) portion of a peripheral nerve.

The micro-transponder may operate as an autonomous wireless unit, capable of detecting spike signals generated by peripheral nerves and relaying such signals to external receivers for further processing. It should be understood that the micro-transponder performs such operations while being powered by external RF electromagnetic signals. The above-mentioned capabilities are facilitated by the fact that magnetic fields are not readily attenuated by human tissue. This enables the RF electromagnetic signals to sufficiently penetrate the human body so that signals can be received and/or transmitted by the micro-transponder. In other words, the micro-coils 922 are adapted to magnetically interact with the RF field whose magnetic flux fluctuates within the space encompassed by the coils 922. By virtue of being inductors, the coils 922 convert the fluctuations of the magnetic flux of the external RF field into alternating electrical currents, flowing within the coils 922 and the micro-transponder 900. The alternating current is routed, for example, via the coils 922 into the rectifier 914, which is adapted to convert the alternating current into direct current. The direct current may then be used to charge the capacitor 918 thereby creating a potential difference across the JFET of the sensor trigger 916.

In an exemplary embodiment, a gate of the spike sensor JFET 916 may be coupled via the neural spike electrode 919 to the neural transmission tissue, such as neurons. The gate of the spike sensor JFET 16 may be chosen to have a threshold voltage that is within a voltage range of those signals produced by the neural axons. In this manner, during spike phases of the neural axons, the gate of the spike sensor JFET 916 becomes open, thereby closing the circuit 910.

Once the circuit 910 closes, the external RF electromagnetic field generates an LC response in the coupled inductor 922 and capacitor 918, which then resonate with the external RF electromagnetic field with its resonance matching the modulating frequency of the RF electromagnetic field.

The LC characteristic of the circuit 910, as well as the threshold voltage of the gate of spike sensor JFET 916 can be chosen to determine a unique modulation within the coupled inductor 922 and capacitor 918 thereby providing a desired ID signal for the micro-transponder. Accordingly, the spike sensor JFET 916 provides the RF identity modulator 917 with a trigger signal for generating desired RF signals. The ID signal may indicate the nature of the neural activity in the vicinity of the micro-transponder as well as the location of the neural activity within the body.

It should be appreciated that the RF capabilities render the micro-transponder 900 a passive device which reacts to incoming carrier RF signals. That is, the micro-transponder 900 does not actively emit any signals but rather reflects and/or scatters the electromagnetic signals of the carrier RF wave to provide signals having specific modulation. In so doing, the micro-transponder 900 draws power from the carrier RF wave for powering the electrical components therein.

While the above mentioned components illustrated in FIG. 9 may be used to receive signals form the micro-transponder in response to spike signals generated by peripheral nerves, other components of the micro-transponder 900 may include components for stimulating the peripheral nerves using the external RF signals. For example, the RF signals received by the coils 922 may be converted to electrical signals, via the RF identity and trigger demodulator 913, so as for providing sufficient current and voltage for stimulating the peripheral nerves. Hence, the RF identity and trigger demodulator 913 derives power from an RF carrier signal for powering the stimulus driver 920, which delivers electrical signals suitable for stimulating neural conduction tissue, such as axons. This may be used to treat nerves that are damaged or that are otherwise physiologically deficient.

It should be understood that, in certain embodiments, the minimum size for the micro-transponders may be limited by the size of the micro-coil responsible for power induction, and secondarily by the size of the capacitors necessary for tuning power storage and timing. In fact, micro-coils less than 1 millimeter (mm) in diameter and just a few micrometers thick can provide sufficient wireless power to operate the complex micro-electronics that can be manufactured on integrated circuit chips that are typically much smaller than these coils. Combining the sophisticated functionality of micro-electronic chips with the wireless performance of these micro-coils creates the smallest possible, minimally invasive implants, in the form of tiny flecks as small as ˜0.1 mm thick and ˜1 mm wide. These size and power advantages make it possible to add relatively complex digital electronics to the smallest, least invasive micro-transponder implant.

FIG. 9 is a functional schematic of a complete micro-transponder for sensing and/or stimulating neural activity, in accordance with one embodiment. The circuit is designed for dependent triggering operation (synchronous stimulation). The circuit 900 includes electrical components adapted to electrically interface with neurons of peripheral nerves. The circuit 900 further includes electrical components which enable the micro-transponder to wirelessly interact with systems external to the micro-transponder. Such systems may include other transponders implanted within the body or external coils and/or a receiver. The wireless capabilities of the circuit 900 enable the delivery of electrical signals to and/or from the peripheral nerves. These include electrical signals indicative of neural spike signals and/or signals configured to stimulate peripheral nerves distributed throughout the subcutaneous tissue.

Accordingly, the circuit 900 includes the micro-coil 922 coiled about a central axis 912. The micro-coil 922 is coupled in parallel to a capacitor 911 and to an RF identity modulator 917 via a switch 915. The RF identity modulator 917 is coupled to an RF identity and trigger demodulator 913, which in turn is coupled to a rectifier 914. The rectifier 914 is coupled to a spike sensor trigger 916 and to a stimulus driver 920. The rectifier 914 and the spike sensor 916 are both coupled in parallel to a capacitor 918. In addition, the spike sensor 916 is coupled to a neural spike electrode 919, thereby electrically connecting the spike sensor 916 to neural transmission tissue (neurons). Similarly, the neural stimulus electrode 921 also connects the stimulus driver 920 to neural conduction tissue (axons). The spike sensor 916 is made up of one or more junction field effect transistors (JFET). As will be appreciated by those of ordinary skilled in the art, the JFET may include metal oxide semiconductors field effect transistors (MOSFETS).

The sensors, drivers, and other electronic components described in the present application can be fabricated using standard small scale or very large scale integration (VLSI) methods. Further, the spike sensor 916 is coupled to the RF identity modulator 917, which is adapted to modulate an incoming/carrier RF signal in response to neural spike signals detected by the spike sensor 916. In one embodiment, the neural electrodes (i.e., neural spike electrode 919 and neural stimulus electrode 921) to which the spike sensor 916 and the stimulus driver 920 are connected, respectively, can be bundled and configured to interface with neural conduction (axon) portion of a peripheral nerve.

One configuration of the above components, as depicted by FIG. 9, enables the micro-transponder to operate as an autonomous wireless unit, capable of detecting spike signals generated by peripheral nerves, and relaying such signals to external receivers for further processing. It should be understood that the micro-transponder performs such operations while being powered by external RF electromagnetic signals. The above-mentioned capabilities are facilitated by the fact that magnetic fields are not readily attenuated by human tissue. This enables the RF electromagnetic signals to sufficiently penetrate the human body so that signals can be received and/or transmitted by the micro-transponder. In other words, the micro-coil 922 is designed and configured to magnetically interact with the RF field whose magnetic flux fluctuates within the space encompassed by the micro-coil 922. By virtue of being inductors, the micro-coils 922 convert the fluctuations of the magnetic flux of the external RF field into alternating electrical currents, flowing within the micro-coil 922 and the circuit 910. The alternating current is routed, for example, into the rectifier 914, which converts the alternating current into direct current. The direct current may then be used to charge the capacitor 918, thereby creating a potential difference across the JFET of the spike sensor 916.

In an exemplary embodiment, a gate of the spike sensor 916 JFET may be coupled via the neural spike electrode 919 to the neural transmission tissue (neurons). The gate of the spike sensor 916 JFET may be chosen to have a threshold voltage that is within a voltage range of those signals produced by the neural axons. In this manner, during spike phases of the neural axons, the gate of the spike sensor 916 becomes open, thereby closing the circuit 910. Once the circuit 910 closes, the external RF electromagnetic field generates an LC response in the coupled inductor 922 and capacitor 918, which then resonate with the external RF electromagnetic field, with its resonance matching the modulating frequency of the RF electromagnetic field. The LC characteristic of the circuit 910, as well as the threshold voltage of the gate of spike sensor 916 JFET, can be chosen to determine a unique modulation within the coupled micro-coil (i.e. inductor) 922 and capacitor 918, thereby providing a identifying signal for the micro-transponder. Accordingly, the spike sensor 16 JFET provides the RF identity modulator 917 with a unique trigger signal for generating desired RF signals. The identity signal may indicate the nature of the neural activity in the vicinity of the micro-transponder, as well as the location of the neural activity within the body as derived from the specific identified micro-transponder position.

It should be appreciated that the RF capabilities, as discussed above with respect to the circuit 910, can render the micro-transponder a passive device which reacts to incoming carrier RF signals. That is, the circuit 910 does not actively emit any signals, but rather reflects and/or scatters the electromagnetic signals of the carrier RF wave to provide signals having specific modulation. In so doing, the circuit 910 draws power from a carrier RF wave to power the electrical components forming the circuit 910.

While the above-mentioned components illustrated in FIG. 9 may be used to receive signals from the micro-transponder in response to spike signals generated by peripheral nerves, other components of circuit 910 of the micro-transponder may include components for stimulating the peripheral nerves using the external RF signals. For example, the RF signals received by the micro-coil 922 may be converted to electrical signals, via the RF identity and trigger demodulator 913, so as to provide sufficient current and voltage for stimulating the peripheral nerves. Hence, the RF identity and trigger demodulator 913 derives power from an RF carrier signal for powering the stimulus driver 920, which delivers electrical signals suitable for stimulating neural conduction tissue (axons). This may be used to treat nerves that are damaged or that are otherwise physiologically deficient. Because of the nature of the identifying signal, a micro-transponder can be selectively activated to provide electrostimulation.

It should be understood that, in certain embodiments, the minimum size for the micro-transponders may be limited by the size of the micro-coil responsible for power induction, and secondarily by the size of the capacitors necessary for tuning power storage and timing. Therefore, micro-coil designs that minimize the complex integrated circuits can be fabricated to an extremely small size (such as less than 1 micron) and ultra-low power technology. The size and power advantages make it possible to add relatively complex digital electronics to the smallest transponder.

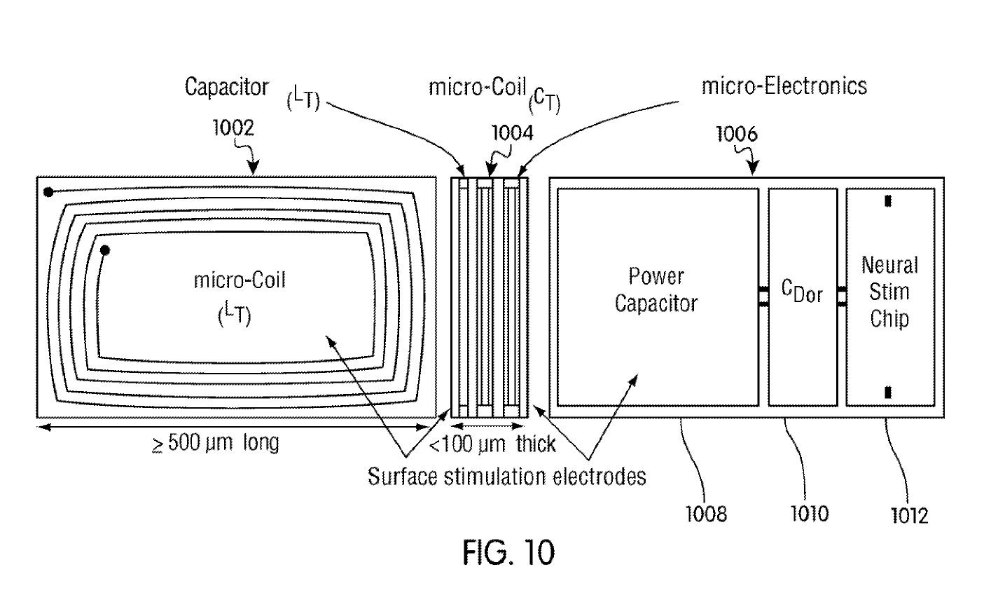

FIG. 10 is an illustration of a laminar spiral micro-foil used in the construction of a micro-transponder platform for stimulating neural activity, in accordance with one embodiment. The spiral antenna may be a flat spiral antenna, a two-layered spiral antenna, a split-spiral antenna or any other suitable configuration. As depicted, herein, the micro-transponder includes a laminar spiral micro-coil (LT) 1002 coupled to a capacitor (CT) 1004 which in turn is coupled to a microelectronics chip 1006. The micro-electronics chip 1006 includes a power capacitor element 1008 coupled to a capacitor (CDUR) element 1010, which in turn is coupled to a neural stimulation chip element 1012.

In an exemplary embodiment of the micro-transponder platform, the micro-coil is no more than 500 micrometers long by 500 micrometers wide and the combined thickness of the laminar spiral micro-coil (LT) 1002, capacitor (CT) 1004 and micro-electronics chip 1006 is no more than 100 micrometers.

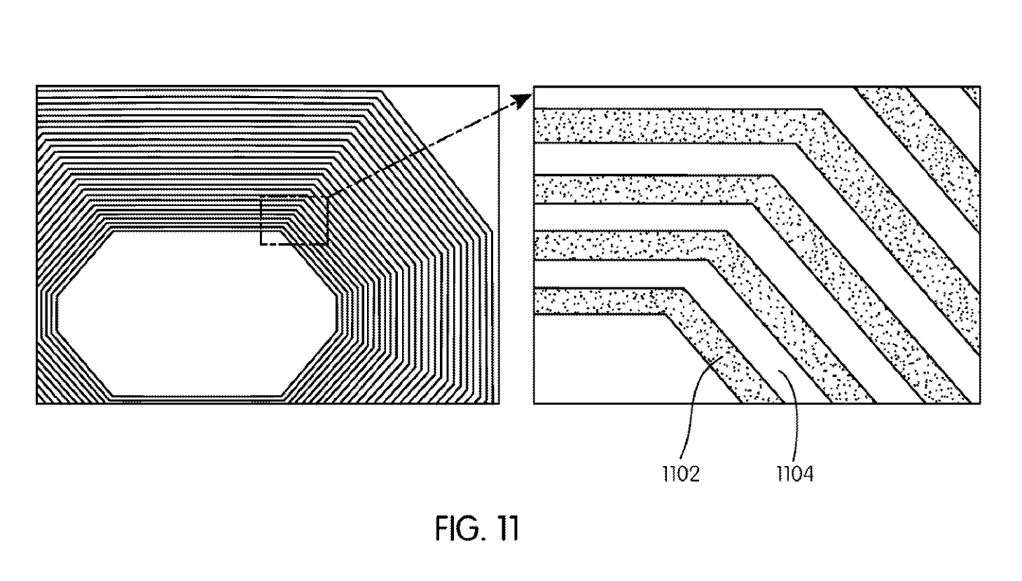

FIG. 11 is an illustration of a gold laminar spiral micro-coil electroplated onto a substrate, in accordance with one embodiment. As depicted in the photomicrographs, gold conductor lines are initially electroplated in a tight spiral pattern onto a non-reactive substrate (e.g., glass, silicon, etc.) In one embodiment, the gold laminar spiral micro-coil can include gold conductor lines 1102 that are about 10 micrometers wide and the spacing 1104 between the conductor lines set at about 10 micrometers. In another embodiment, the gold laminar spiral micro-coil can include gold conductor lines 1102 that are about 20 micrometers wide and the spacing 1104 between the conductor lines set at about 20 micrometers. It should be understood, however, that the widths of the gold conductor line 1102 and line spacing 1104 between them can be set to any value as long as the resulting micro-coil can produce the desired induced current for the desired application.

In certain embodiments, once the gold spiral micro-coil has been electroplated onto the substrate, a polymer-based layer is spun on top of the micro-coils to provide a layer of protection against corrosion and decay once implanted. Long term studies of animals with SU-8 implants have verified the bio-compatibility of SU-8 plastic by demonstrating that these SU-8 implants remain functional without signs of tissue reaction or material degradation for the duration of the studies. Therefore, typically, the polymer-based layer is comprised of an SU-8 or equivalent type of plastic having a thickness of approximately 30 micrometers.

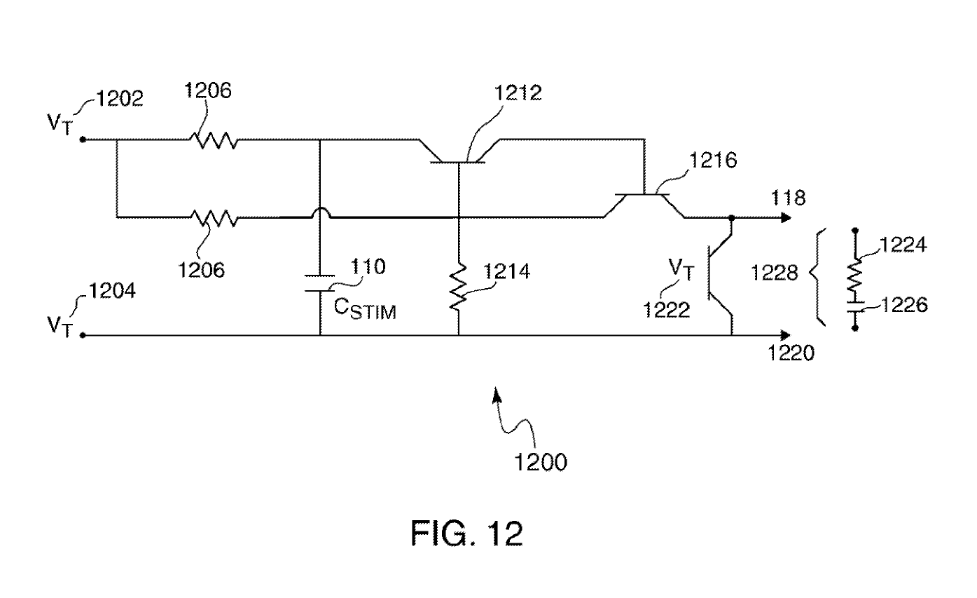

With reference to FIG. 12, a schematic diagram depicts a depolarizing micro-transponder driver circuit 1200 in accordance with an embodiment. An oscillating trigger voltage (VT and −VT) may be applied between the input nodes 1202 and 1204 of the driver circuit 1200. An auto-triggering micro-transponder may employ a bi-stable switch 1212 to oscillate between the charging phase that builds up a charge on the stimulus capacitor CSTIM 1210 and the discharge phase that can be triggered when the charge reaches the desired voltage and closes the switch 1212 to discharge the capacitor 1210 through stimulus electrodes 1218 and 1220.

A resistor 1206 regulates the stimulus frequency by limiting the charging rate. The stimulus peak and amplitude are largely determined by the effective tissue resistance 1228, modeled with a resistance 1224 and a capacitance 1226. As such, the stimulus is generally independent of the applied RF power intensity. On the other hand, increasing the RF power may increase the stimulation frequency by reducing the time it takes to charge up to the stimulus voltage.

When a stimulation signal is applied to living tissue at frequencies higher than two hertz, the tissue typically becomes polarized, exhibiting an inherent capacitance 1226 by storing a persistent electrical charge. In order to reduce the polarization effect, a depolarization switch 1222 is connected between the electrodes 1218 and 1220. The gate terminal of the depolarization switch 1222 is connected to the oscillating trigger voltage VT, so that once each cycle, the depolarization switch shorts the electrodes 1218 and 1220 and reduces the charge stored in the inherent tissue capacitance 1226. The timing of the depolarization switch 122 permits the stimulation pulse to be substantially discharged before the depolarization switch 122 closes and shorts the electrodes 118 and 120. Similarly, the depolarization switch 122 is timed to open before a subsequent stimulation pulse arrives. The timing of the depolarization switch 122 may be generated relative to the timing of the stimulation pulse, The timing may be accomplished using digital delays, analog delays, clocks, logic devices or any other suitable timing mechanism.

A simple zener diode component may be included in a stimulator circuit as presented in FIG. 1. Asynchronous stimulations can be accomplished using the zener diode to accomplish voltage levels for auto-triggering.

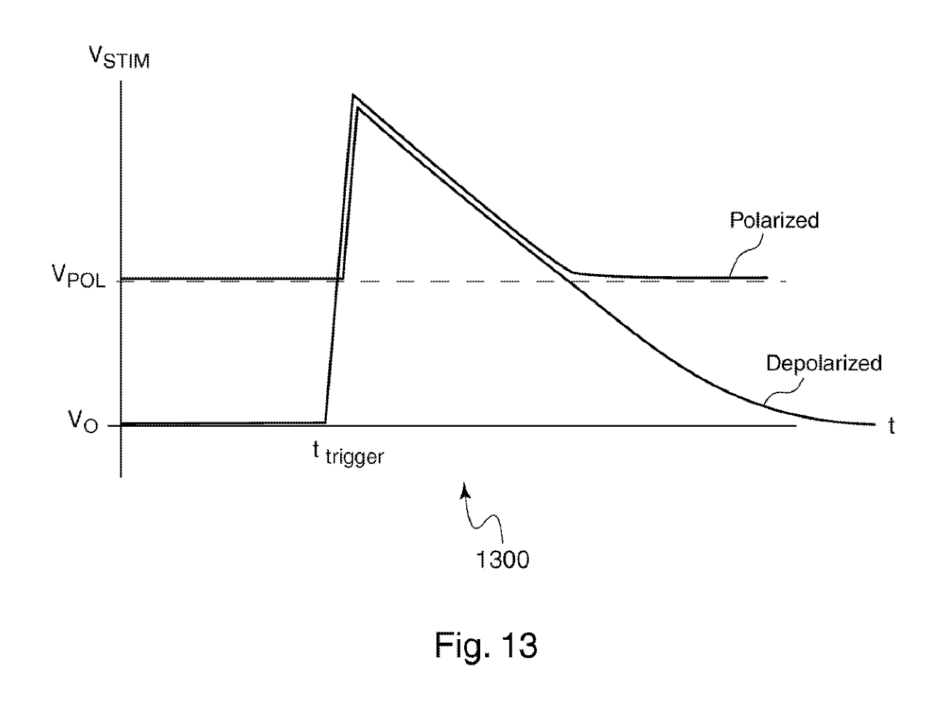

With reference to FIG. 13, a graph depicts an exemplary stimulus discharge in accordance with an embodiment. When a trigger signal is received, the stimulus capacitor discharges current between the electrodes. Depending on the tissue resistance, the voltage quickly returns to a rest voltage level at approximately the initial voltage level. When the frequency of the trigger signal is increased, a polarization effect causes the rest voltage to rise to a polarization voltage above the initial voltage. With a depolarization switch between the electrodes, each trigger signal causes the rest voltage to be re-established and lowered to about the initial voltage level.

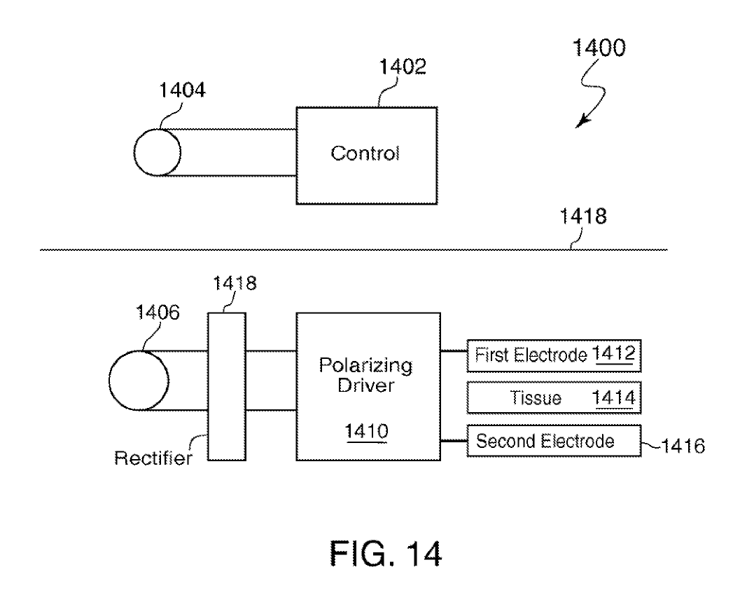

With reference to FIG. 14, a block diagram depicts a depolarizing micro-transponder system 1400 in accordance with an embodiment. A control component energizes an external resonator element 1404 positioned externally relative to an organic layer boundary 1418. Energized, the external resonator element 1404 resonates energy at a resonant frequency, such as a selected RF. Internal resonator element 1406, positioned internally relative to an organic layer boundary 1418, is tuned to resonate at the same resonant frequency, or a harmonically related resonant frequency as the external resonator element 1404. Energized by the resonating energy, the internal resonator element 1406 generates pulses of energy rectified by a rectifier 1418. The energy may typically be stored and produced subject to timing controls or other forms of control. The energy is provided to the depolarizing driver 1410. A first electrode 1412 is polarized relative to a second electrode 1416 so that current is drawn through the tissue 1414 being stimulated, proximate to the electrode 1412 and 1416. The first electrode 1412 is polarized relative to the second electrode 1416 in the opposite polarization to draw an oppositely directed current through the tissue 1414, depolarizing the tissue 1414. The electrodes 1412 and 1416 may be typically made of gold or a platinum iridium alloy, or any other suitable material.

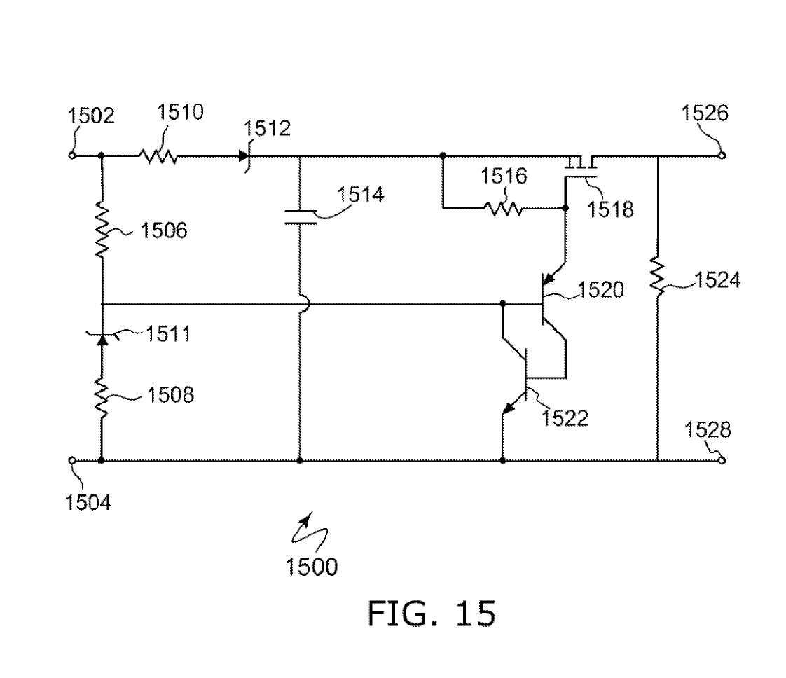

With reference to FIG. 15, a circuit diagram depicts a depolarization driver circuit 1500, in accordance with an embodiment. A trigger signal is applied between electrodes 1502 and 1504. A charge capacitance 1514 is charged on the charge capacitance 1514. Schottky diode 1512 prevents the backflow of stimulus charge during the trigger phase. The charge rate is regulated by resistances 1510, 1506 and 1508. Resistances 1506 and 1508 form a voltage divider so that a portion of the trigger signal operate the bipolar switches 1520 and 1522. The trigger signal closes CMOS 1518 through resistance 1516, connecting the pulse between electrodes 1526 and 1528. A depolarization resistance 1524 is connected between the electrodes 1526 and 1528 to balance the charge stored in the tissue between the electrodes 1526 and 1528 between pulses. The specific breakdown voltage of the optional Zener diode 1511 provides for auto-triggering setting the upper limit of the voltage divider, at which point the bipolar switches are triggered by any further increase in the stimulus voltage. In addition to providing this auto-triggering feature for the purpose of asynchronous stimulation, the particular breakdown voltage of this Zener diode 1511 sets the maximum stimulus voltage. Otherwise the stimulus voltage is a function of the RF power level reaching the transponder from the external reader coil when the stimulus is triggered.

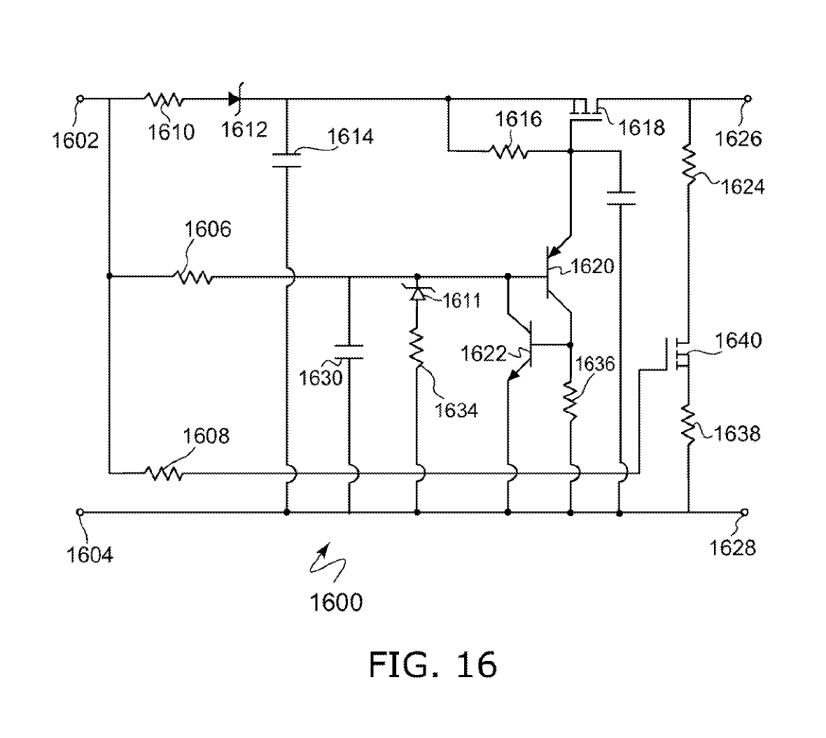

With reference to FIG. 16, a circuit diagram depicts a depolarization driver circuit 1600, in accordance with an embodiment. A trigger signal is applied between electrodes 1602 and 1604. A charge capacitance 1614 is charged on the charge capacitance 1614. Schottky diode 1612 prevents the backflow of stimulus charge during the trigger phase. The charge rate is regulated by resistances 1610, 1606, 1634 and 1608. Resistances 1606 and 1608 form a voltage divider so that a portion of the trigger signal operate the bipolar switches 1620 and 1622. The trigger signal closes CMOS 1618 through resistance 1616, connecting the pulse between electrodes 1626 and 1628. Depolarization resistances 1624 and 1638 are connected to a depolarization CMOS 1640 between the electrodes 1626 and 1628 to balance the charge stored in the tissue between the electrodes 1626 and 1628 between pulses. The specific breakdown voltage of the optional Zener diode 1611 provides for auto-triggering setting the upper limit of the voltage divider, at which point the bipolar switches are triggered by any further increase in the stimulus voltage. In addition to providing this auto-triggering feature for the purpose of asynchronous stimulation, the particular breakdown voltage of this Zener diode 1611 sets the maximum stimulus voltage. Otherwise the stimulus voltage is a function of the RF power level reaching the transponder from the external reader coil when the stimulus is triggered.

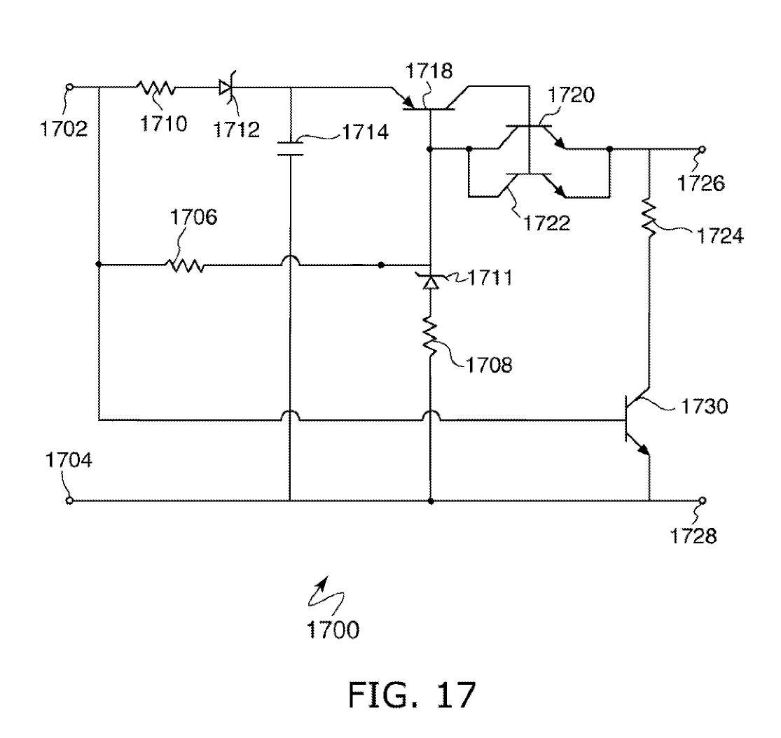

With reference to FIG. 17, a circuit diagram depicts a depolarization driver circuit 1700, in accordance with an embodiment. A trigger signal is applied between electrodes 1702 and 1704. A charge capacitance 1714 is charged on the charge capacitance 1714. Schottky diode 1712 prevents the backflow of stimulus charge during the trigger phase. The charge rate is regulated by resistances 1710, 1706 and 1708. Resistances 1706 and 1708 form a voltage divider so that a portion of the trigger signal operate the bipolar switches 1720 and 1722. The trigger signal closes switch 1718 through resistance 1716, connecting the pulse between electrodes 1726 and 1728. A depolarization resistance 1724 is connected to a bipolar switch 1730 between the electrodes 1726 and 1728 to balance the charge stored in the tissue between the electrodes 1726 and 1728 between pulses. The specific breakdown voltage of the optional Zener diode 1711 provides for auto-triggering setting the upper limit of the voltage divider, at which point the bipolar switches are triggered by any further increase in the stimulus voltage. In addition to providing this auto-triggering feature for the purpose of asynchronous stimulation, the particular breakdown voltage of this Zener diode 1711 sets the maximum stimulus voltage. Otherwise the stimulus voltage is a function of the RF power level reaching the transponder from the external reader coil when the stimulus is triggered.

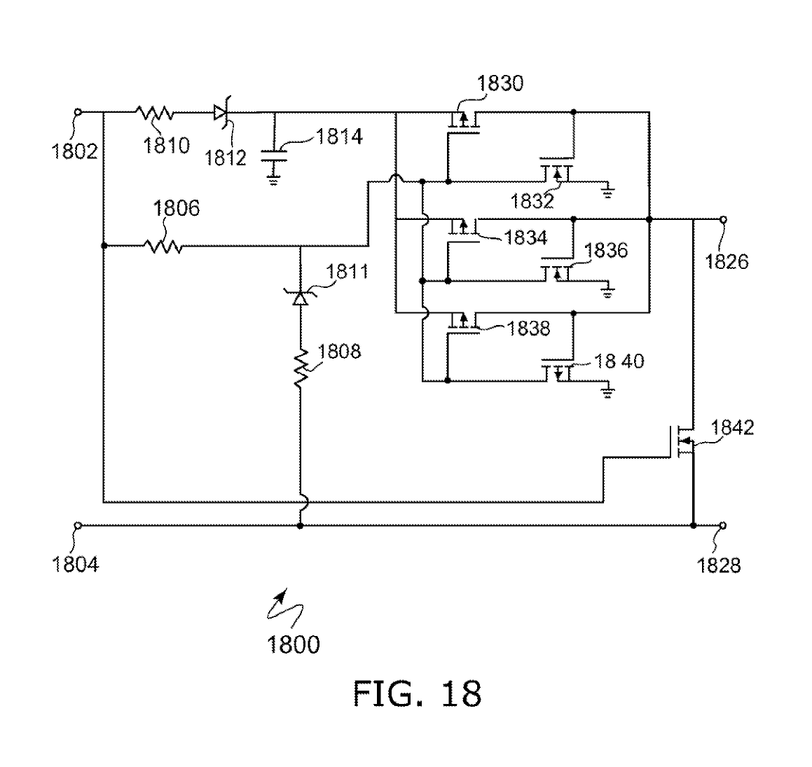

With reference to FIG. 18A, a circuit diagram depicts a depolarization driver circuit 1800, in accordance with an embodiment. A trigger signal is applied between electrodes 1802 and 1804. A charge capacitance 1814 is charged on the charge capacitance 1814. Schottky diode 1812 prevents the backflow of stimulus charge during the trigger phase. The charge rate is regulated by resistances 1810, 1806 and 1808. Resistances 1806 and 1808 form a voltage divider so that a portion of the trigger signal operate the CMOS switches 1830, 1832, 1834, 1836, 1838 and 1840. The trigger signal closes CMOS 1830, 1834 and 1836 connecting the pulse between electrodes 1826 and 1828. A depolarization CMOS 1842 is connected between the electrodes 1826 and 1828 to balance the charge stored in the tissue between the electrodes 1826 and 1828 between pulses. The specific breakdown voltage of the optional Zener diode 1811 provides for auto-triggering setting the upper limit of the voltage divider, at which point the bipolar switches are triggered by any further increase in the stimulus voltage. In addition to providing this auto-triggering feature for the purpose of asynchronous stimulation, the particular breakdown voltage of this Zener diode 1811 sets the maximum stimulus voltage. Otherwise the stimulus voltage is a function of the RF power level reaching the transponder from the external reader coil when the stimulus is triggered.

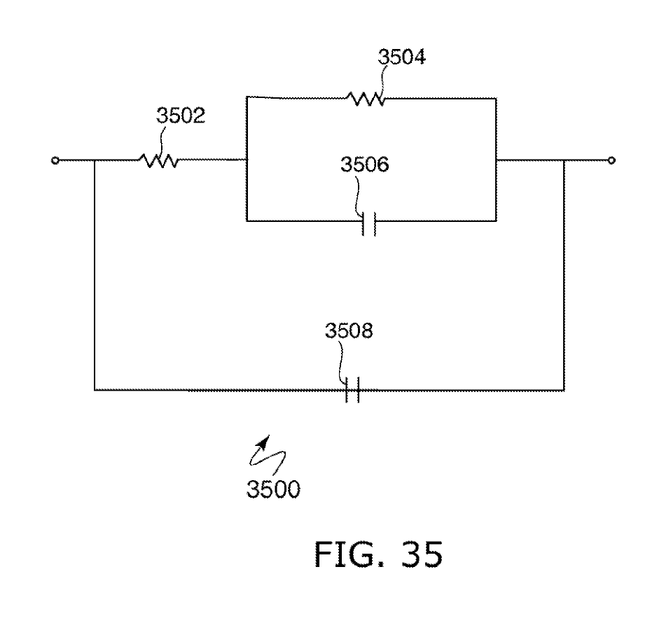

With reference to FIG. 35, a circuit diagram depicts a tissue model. Depolarization becomes important because the tissue behaves as a non-linear load that can be modeled as shown. A resistance 3502 is in series with a resistance 3504 in parallel with a capacitance 3506. This arrangement is parallel to a second capacitance 3508. The capacitances 3506 and 3508 result in charge being stored in the circuit when an intermittent signal is applied, as happens in the tissue being stimulated by intermittent stimulation signals.

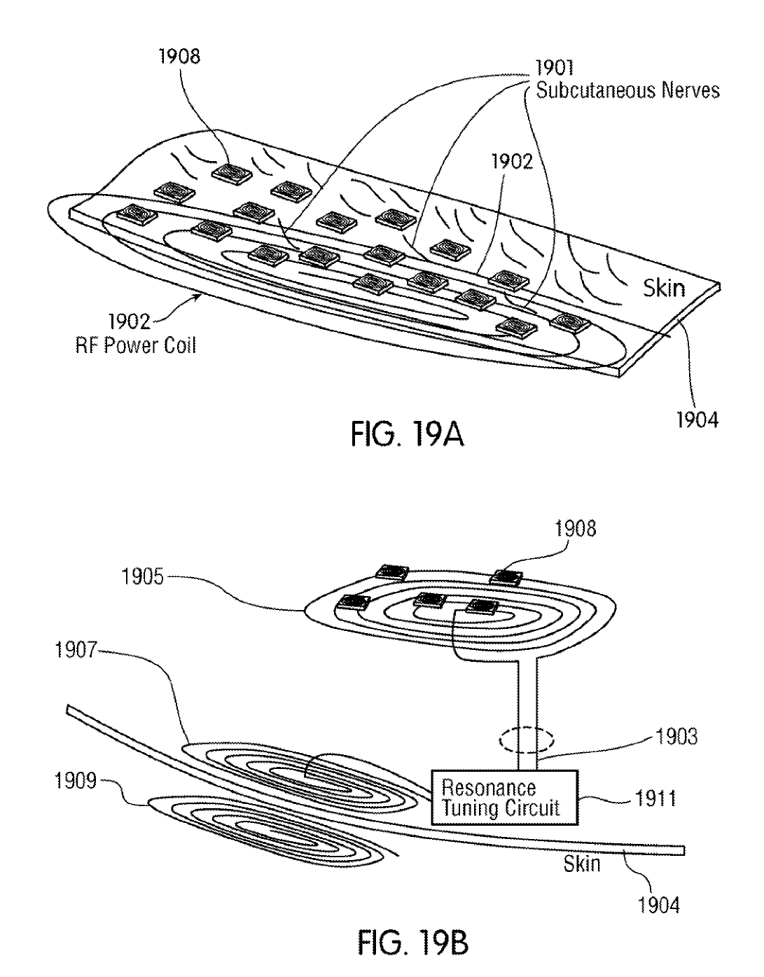

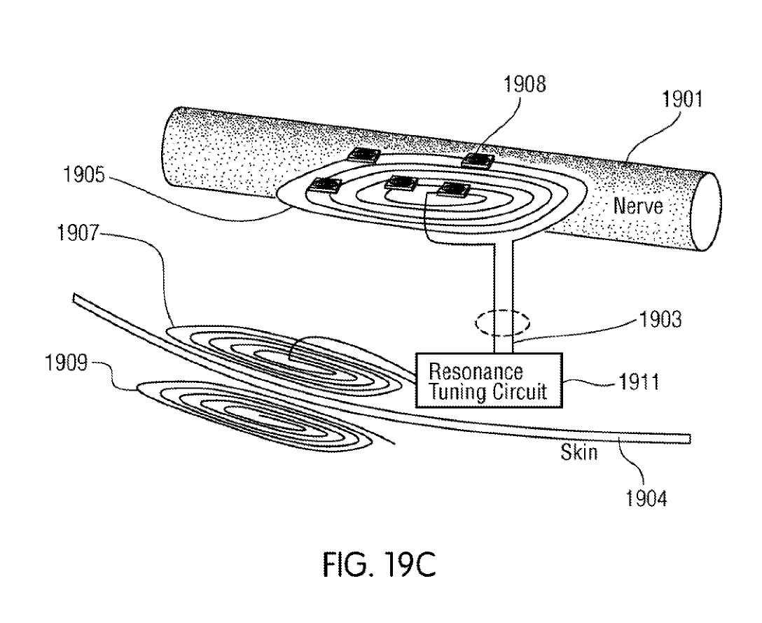

FIG. 19A is an illustration of a deployment of a plurality of wireless micro-transponders distributed throughout subcutaneous vascular beds and terminal nerve fields, in accordance with one embodiment. As depicted, a plurality of independent wireless micro-transponders 1908 are implanted subcutaneously in a spread pattern under the skin 1904 over the area that is affected by the chronic pain.

Each micro-transponder is positioned proximate to and/or interfaced with a branch of the subcutaneous sensory nerves 1901 to provide electrostimulation of those nerves. In one embodiment, only synchronous micro-transponders are deployed. In another embodiment only asynchronous micro-transponders are deployed. In yet another embodiment a combination of synchronous and asynchronous micro-transponders are deployed.

After the deployment of the micro-transponders, electrostimulation can be applied by positioning a RF power coil 1902 proximate to the location where the micro-transponders are implanted. The parameters for effective electrostimulation may depend upon several factors, including: the size of the nerve or nerve fiber being stimulated, the effective electrode/nerve interface contact, the conductivity of the tissue matrix, and the geometric configuration of the stimulating fields. While clinical and empirical studies have determined a general range of suitable electrical stimulation parameters for conventional electrode techniques, the parameters for micro-scale stimulation of widely distributed fields of sensory nerve fibers are likely to differ significantly with respect to both stimulus current intensities and the subjective sensory experience evoked by that stimulation.

Parameters for effective repetitive impulse stimulation using conventional electrode techniques are typically reported with amplitudes ranging from up to about 10 V (or up to about 1 mA) lasting up to about 1 millisecond repeated up to about 100 pulses/s for periods lasting several seconds to a few minutes at a time. In an exemplary embodiment, effective repetitive impulse stimulation can be achieved with an amplitude of less than 100 ILIA and stimulation pulses lasting less than 100 gs.

FIG. 19B is an illustration of a deployment of wireless micro-transponders to enable coupling with deep micro-transponder implants, in accordance with one embodiment. As shown herein, two simple electrical wires 1903 lead from the subdermal/subcutaneous coil 1907 to the deeper site where a field of micro-transponders 1908 are implanted. Threading the wires 1903 through the interstitial spaces between muscles and skin involves routine minimally invasive surgical procedures as simple as passing the lead through hypodermic tubing, similar to routine endoscopic methods involving catheters. The minimal risks of such interstitial wires 1903 are widely accepted.

A deep coil 1905 is implanted to couple with the deeply implanted field of micro-transponders 1908 located near deep targets of micro-stimulation, such as deep peripheral nerves, muscles or organs such as the bladder or stomach as needed to treat a variety of clinical applications. The deep coil 1905 is tuned to extend the resonance of the external coil 1909 to the immediate vicinity of the implanted micro-transponders 1908 for maximal coupling efficiency. In addition to extending the effective range of the micro-transponder 1908 implants, the deep coil 1905 also provides another wireless link that can preserve the integrity of any further protective barrier around the target site. For instance, the deep coil 1905 can activate micro-transponders 1908 embedded within a peripheral nerve without damaging the epineurium that protects the sensitive intraneural tissues. To ensure optimal tuning of the transfer coils (e.g., the subdermal coil 1907) a variable capacitor or other tuning elements in a resonance tuning circuit 1911 are added to the subdermal coil 1907 where it can be implanted with minimal risk of tissue damage.

FIG. 19C is an illustration of a deployment of wireless micro-transponders to enable coupling with deep neural micro-transponder implants, in accordance with one embodiment. As shown herein, an extraneural interface coil 1905 positioned proximate to (or interfaced with) a nerve fiber or cell cluster 1901 is interconnected to a subcutaneous relay coil 1907 by a simple pair of leads 1903 that mediate all the signals and power necessary to operate micro-transponders 1908 implanted anywhere in the body, beyond the direct effective range of any external coil 1909 (e.g., epidermal coil, etc.). In addition to extending the effective range of the micro-transponder 1908 implants, the deep coil 1905 also provides another wireless link that can preserve the integrity of any further protective barrier around the target site. For instance, the deep coil 1905 can activate micro-transponders 1908 embedded within a peripheral nerve without damaging the epineurium that protects the sensitive intraneural tissues. In certain embodiments, the subdermal relay coil 1907 is tuned to the external coil 1909 and implanted immediately under the external coil 1909 just below the surface of the skin 1904 for maximum near-field wireless magnetic coupling. This allows the RF waves generated by the external coil 1909 to penetrate the body without long-term damage to the skin 1904 and the risk of infection. In other embodiments, the subdermal relay coil 1907 is tuned to the external coil 1909 and implanted deeper in the tissue subcutaneously.

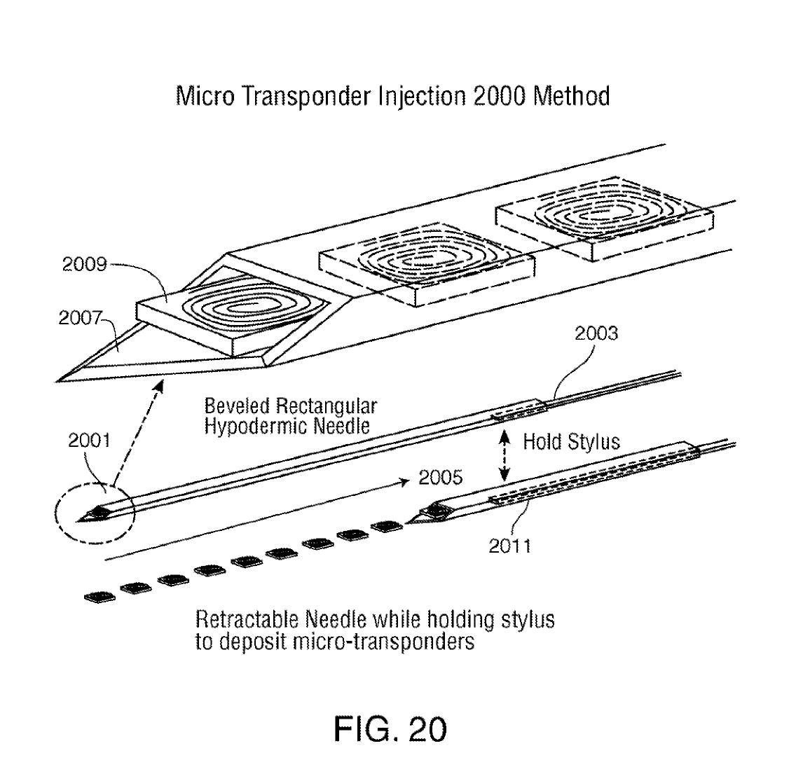

FIG. 20 shows an example injection system 2000 comprising a loaded cannula 2005, stylet 2003 that can push through the cannula 2005. To safely insert a micro-stimulator/micro-transponder to a body location cannula 2005 is designed to be in square and small diameter as the introducer with tapered dilator that does not have sharp edges. The front tip 2001 of Cannula 2005 may include an extruded edge 2007 that guides loaded micro-transponders 2009 into a target body location where the placement of micro-transponders or array of micro-transponders will likely be a drop-down placement. Micro-transponders are deposited while pushing through stylet 2003 and retracting the needle/cannula 2005.

Cannula 2005 may also have the ability to retrieve a micro device array immediately or during the next 8-10 days, without a cut-down or reinserting another.

FIG. 20 is an illustration of how wireless micro-transponders can be implanted using a beveled rectangular hypodermic needle, in accordance with one embodiment. As shown, the needle 2005 is curved to conform to the transverse cervical curvature (bevel concave) and without further dissection is passed transversely in the subcutaneous space across the base of the affected peripheral nerve tissue. Rapid insertion usually negates the need for even a short active general anesthetic once the surgeon becomes familiar with the technique. Following the placement of the micro-transponders 2009 into the needle 2005, the needle 2005 is carefully withdrawn and the electrode placement and configuration is evaluated using intraoperative testing. Electrostimulation is applied using a temporary RF transmitter placed proximate to the location where the micro-transponders 1003 are implanted, so the patient can report on the stimulation location, intensity, and overall sensation.

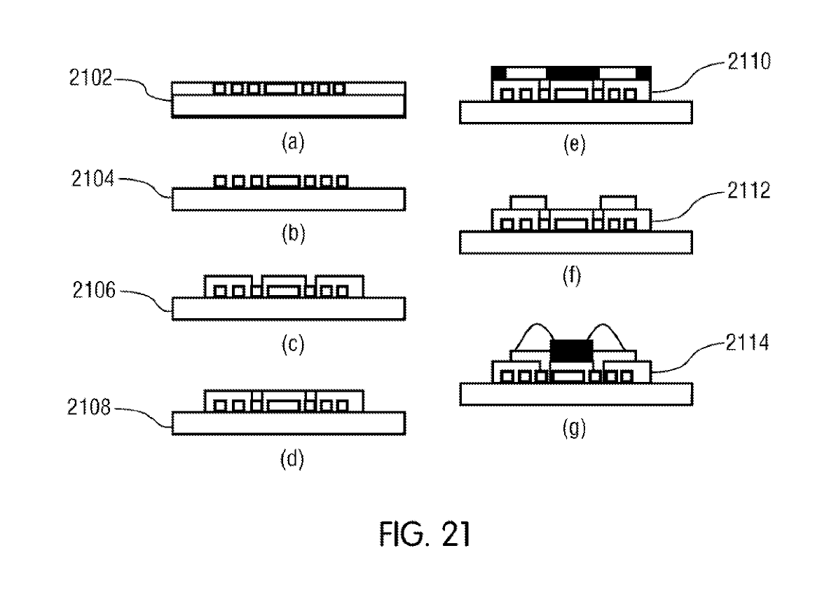

FIG. 21 is an illustration of a fabrication sequence for spiral type wireless micro-transponders, in accordance with one embodiment. At step 2102, a layer of gold spiral coil is electroplated onto a substrate (typically a Pyrex® based material, but other materials may also be used as long as they are compatible with the conducting material used for the spiral coil and the particular application that the resulting micro-transponder will be applied to). Electroplated gold is used as the conductor material due to its high conductivity, resistance to oxidation, and proven ability to be implanted in biological tissue for long periods of time. It should be appreciated, however, that other conducting materials can also be used as long as the material exhibits the conductivity and oxidation resistance characteristics required by the particular application that the micro-transponders would be applied to. Typically, the gold spiral coil conductors have a thickness of between approximately 5 lam to approximately 25 gm.

In one embodiment, the gold spiral coil takes on a first configuration where the gold conductor is approximately 101 m wide and there is approximately 10 lam spacing between the windings. In another embodiment, the gold spiral coil takes on a second configuration where the gold conductor is approximately 20 lam wide and there is approximately 20 lam spacing between the windings. As will be apparent to one of ordinary skill in the art, however, the scope of the present invention is not limited to just these example gold spiral coil configurations, but rather encompasses any combination of conductor widths and winding spacing that are appropriate for the particular application that the coil is applied to.

In step 2104, the first layer of photoresist and the seed layer are removed. In one embodiment, the photoresist layer is removed using a conventional liquid resist stripper to chemically alter the photoresist so that it no longer adheres to the substrate. In another embodiment, the photoresist is removed using a plasma ashing process.

In step 2106, an isolation layer of SU-8 photo resist is spun and patterned to entirely cover each spiral inductor. Typically, the SU-8 layer has a thickness of approximately 30 lam. In step 2108, a top seed layer is deposited on top of the SU-8 isolation layer using a conventional physical vapor deposition (PVD) process such as sputtering. In step 2110, a top layer of positive photoresist coating is patterned onto the top see layer and the SU-8 isolation layer, and in step 2112, a layer of platinum is applied using a conventional electroplating process. In step 2114, a chip capacitor and a RFID chip are attached to the platinum conducting layer using epoxy and making electrical connections by wire bonding. In certain embodiments, the capacitor has a capacitance rating value of up to 10,000 picofarad (pF).

It is possible to implant such small micro-transponders by simply injecting them into the subcutaneous tissue. Using local anesthesia at the injection site, the patient may be positioned laterally or prone depending on the incision entry point. The subcutaneous tissues immediately lateral to the incision are undermined sharply to accept a loop of electrode created after placement and tunneling to prevent electrode migration. A Tuohy needle is gently curved to conform to the transverse posterior cervical curvature (bevel concave) and without further dissection is passed transversely in the subcutaneous space across the base of the affected peripheral nerves. Rapid needle insertion usually obviates the need for even a short acting general anesthetic once the surgeon becomes facile with the technique. Following placement of the electrode into the Tuohy needle, the needle is withdrawn and the electrode placement and configuration is evaluated using intraoperative testing.

After lead placement, stimulation is applied using a temporary RF transmitter to various select electrode combinations enabling the patient to report on the table the stimulation location, intensity and overall sensation. Based on prior experience with wired transponders, most patients should report an immediate stimulation in the selected peripheral nerve distribution with voltage settings from 1 to 4 volts with midrange pulse widths and frequencies. A report of burning pain or muscle pulling should alert the surgeon the electrode is probably placed either too close to the fascia or intramuscularly.

An exemplary micro-transponder array preferably is an array of joined micro-transponders. The joined array is made from or coated with biocompatible material that is sufficiently strong to hold the micro-transponders and remain intact during surgical explantation. An advantage of the joined array is that removal of the array is simpler than unjoined micro-transponders, which would be more difficult to locate and individually extract from the integrated mass of adhered tissues. The concept is flexible, as the array may comprise a joined array of any type of implanted medical devices.

The joined array can be made from several types of biocompatible materials. Exemplary synthetic materials suitable for the removable array include silicone elastomers, or silicone hydrogels, and plastics such as SU-8, or parylene-C. Removable arrays may also be constructed using long-lasting biodegradable polymers including natural materials such as protein-based polymers like gelatin, silk or collagen, and sugar-based poly-saccharides like cellulose or agarose. Other suitable biodegradable polymers have been developed specifically for implant construction including poly-glyolic acids (PGA) and poly-lactic acids (PLA). Such construction materials offer a range of strengths, durability and tissue adhesion properties suitable for a variety of specific implant applications. Furthermore, the surface of any array material may be enhanced to promote specific biological properties such as cell/protein adhesion and tissue reactions by coating the implant with a variety of materials widely employed for this purpose including formulations of PEG (polyethylene glycol) such as PEG-PLA, and commercial products such as Greatbatch Biomimetic Coating (U.S. Pat. No. 6,759,388 B1), and Medtronics' Trillium Bio surface.

Biocompatibility of the array is very important. The linked array can include a coating in the form of a monolayer or thin layer of biocompatible material. Advantages that coatings offer include the ability to link proteins to the coating. The linked proteins can limit what cell types can adhere to the array. The coating can prevent protein adsorption, and it does not significantly increase size of the device.

3-D porous materials are meant to encourage cell ingrowth and organization. The 3-D porous material can act as a buffer between the tissue and micro-transponders to prevent reaction micromotion. The potential benefits for implant/tissue integration must be balanced against the addition risks associated with increasing the overall size of the implant with the additional risks associated with increasing the overall size of the implant with the addition of such 3-D materials.

The visibility of the implant may be enhanced by adding brightly colored dyes to the construction materials thereby facilitating visual location of the array within surrounding tissue in case it must be removed. This can include a marker dye incorporated onto, or into, the device globally. A preferred embodiment would employ a fluorescent dye that becomes visible when exposed to appropriate light sources because it offers the advantage of maximum luminescence to such a level that implants may be visible through the skin.

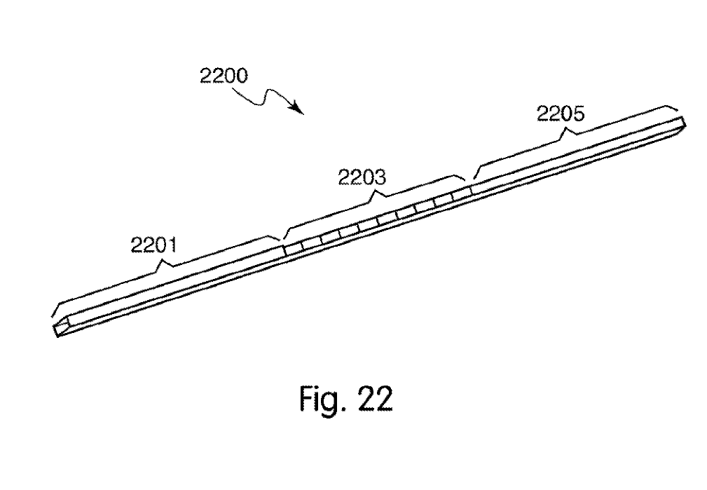

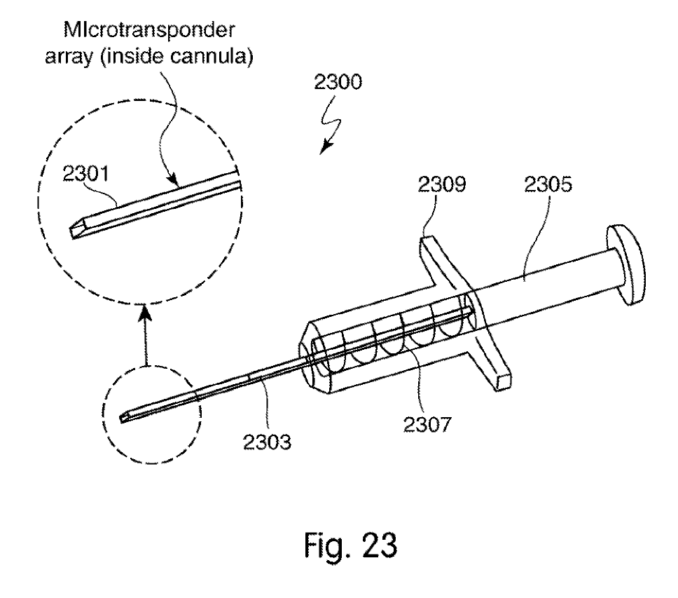

The array of micro-transponders is loaded into the injection system during the manufacturing process. FIG. 22 shows an example of pre-loading micro-transponder array 2203 into Cannula 2201 with or without the attachment of stylet 2205. FIG. 23 shows another example of pre-packaged injection system which has a stylet 2303 attached to syringe-like device where a handle holder 2309, a spring 2307 and a handle 2305 for injection. The whole package is sterilized. Preloaded delivery system may be disposable and used only once. After the manufacturing process is completed, the array 2301 will be ready for implantation after removal from the packaging.

The internal compression spring 2307 will keep the injection system from accidentally dispensing the array during shipment and handling. A needle cap may be used to prevent accidental dispensing and sharps protection.

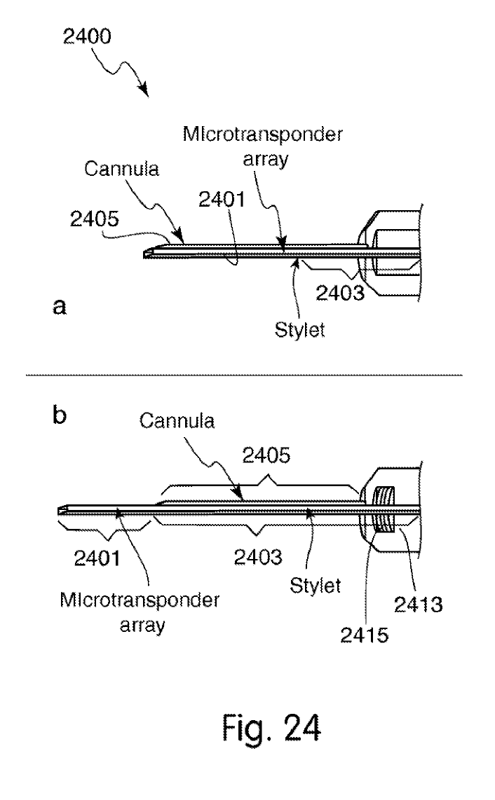

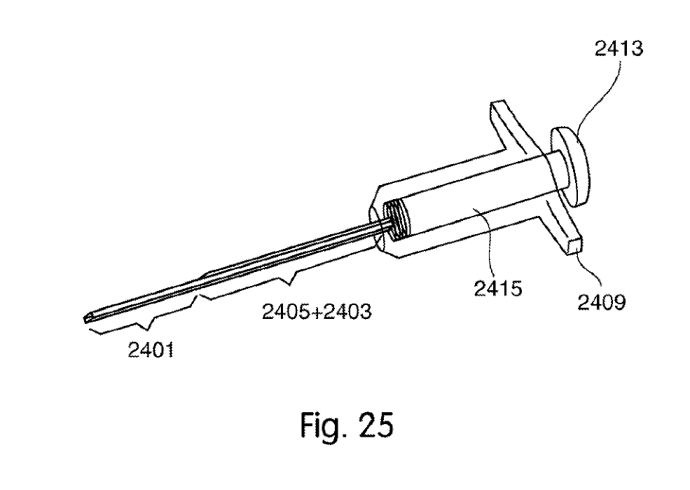

FIG. 24(a) shows a preloaded injection system with a relaxed spring. FIG. 24(b) shows that after inserting the needle/cannula 2405 into the tissue, handle 2413 is pushed compressing the spring 2415 and stylet 2403 and pushing micro-transponder array 2401 into the tissue. After the injection into the tissue, handle holder 2409 is used to retract cannula 2405, leaving the injection array in the tissue. FIG. 25 shows an example look of the injection system immediately after the micro-transponder ejection.

Materials for the construction of the injection system are biocompatible, for example the cannula and stylet can be stainless steel and the handle and the handle holder can be acrylonitrile butadiene styrene (ABS), polycarbonate, or polyurethane. The stylet may also be made of bio-compatible plastics. Sterilization can be conducted and verified according to standard GMP procedure required by FDA for the intended production environment and processes and purposes.

During pre-loading process, the cannula and stylet may need to be fabricated from custom extruded material, so that there is limited space between the array and the walls of the cannula. A biocompatible lubrication material, such as polyethylene glycol (PEG), may be used to reduce the friction between the array and the cannula.

The foreign body response (FBR) is one of the primary modes of failure for electrical implants. Generally this response is triggered by absorbance and denaturation of proteins on the implanted substrate, followed by activation of neutrophils and macrophages. Macrophages that are unable to phagocytose the implant begin fusing to form foreign body giant cells, which release free radicals that may damage the implanted device. Often this is followed by the formation of a fibrous or glial scar which encapsulates the device and segregates it from the target tissue.

It has been shown that both porous scaffold materials and non-fouling coating can reduce the host FBR. A multitude of unique materials and designs have been tested for this purpose. It is desirable to not only reduce the FBR, but also to encourage intimate contact between the implanted devices and target tissues. The primary drawback with previous strategies encouraging tissue integration with implants, is that they can only be removed by excision of actual tissue. This application discloses a novel design to both encourage tissue integration and facilitate removal of devices in the event of failure, patient paranoia, or completion of therapy.

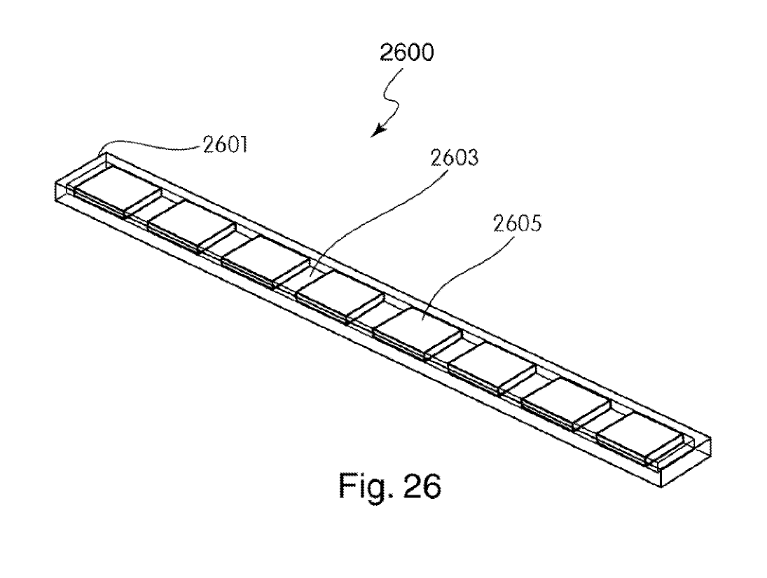

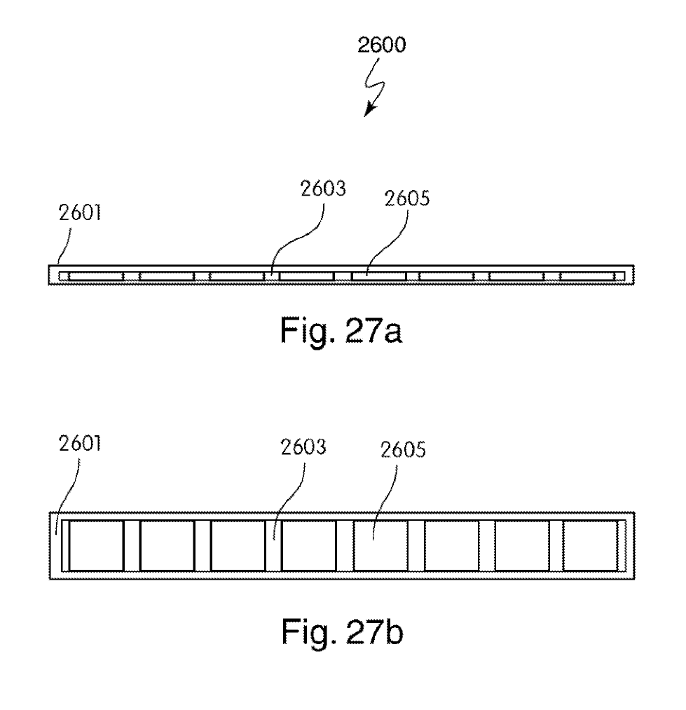

To accomplish this end, as shown in FIGS. 26 and 27, a plurality of individual micro-transponders 2605 can be linked together to form an array and a core strip 2603 by a durable non-fouling material, for example, SU8 with the surface coated with a lubricating, protein adsorption preventing, “stealth” material. The core strip is then embedded within a porous scaffold 2601. The core material will be fabricated from a material (or coated with) that will minimize adhesion with the scaffold and in-growing tissue. Biocompatible material that will encourage growth of surrounding tissue up to the implanted devices and exposed SU8 is used for the scaffold which is designed in a manner to both minimize FBR and encourage the penetration of endothelial cells and neurites. By separating the tissue integrating scaffolding from the solid core, removal of the actual devices can be carried out simply by making an incision to expose the end of the core, grasping it, and then sliding it out from the scaffolding.

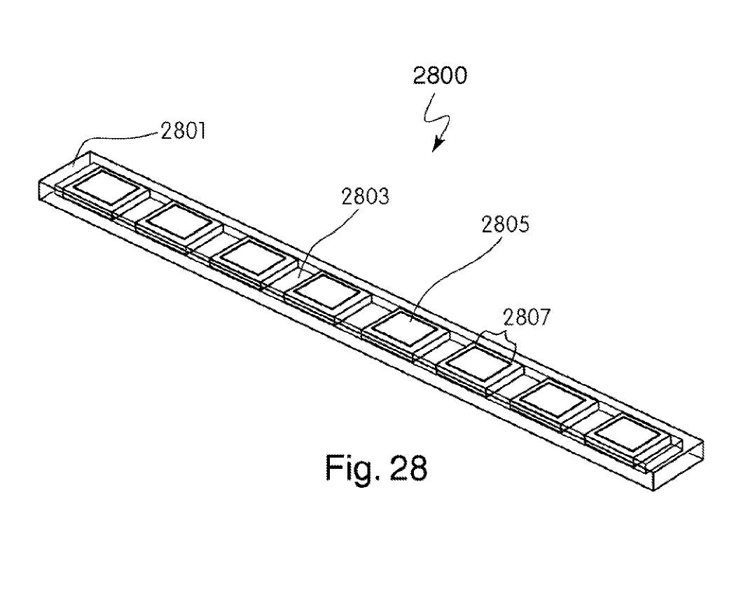

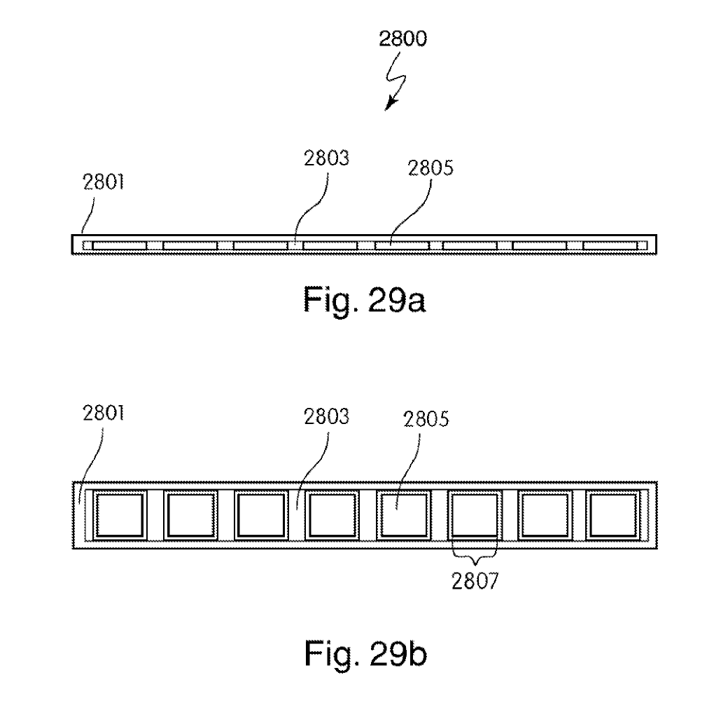

Another embodiment of the micro-transponder array is shown in FIGS. 28 and 29. The core strip 2803 is a strong strip containing an embedded array of individual micro-transponders, where the superior and inferior electrodes of micro-transponders are exposed through “windows” 807. Electrode surfaces and strip may be coated with a lubricious, protein adsorption preventing, “stealth” material. The core strip is then embedded within a porous scaffold/matrix 2801 that the scaffolding will extend into the “windows.” Other durable and more flexible material than SU8 can be used, and embedded micro-transponders can be better protected. Electrodes of micro-transponders 2805 can be totally isolated from proteins/tissues, but still affect ions in solution.

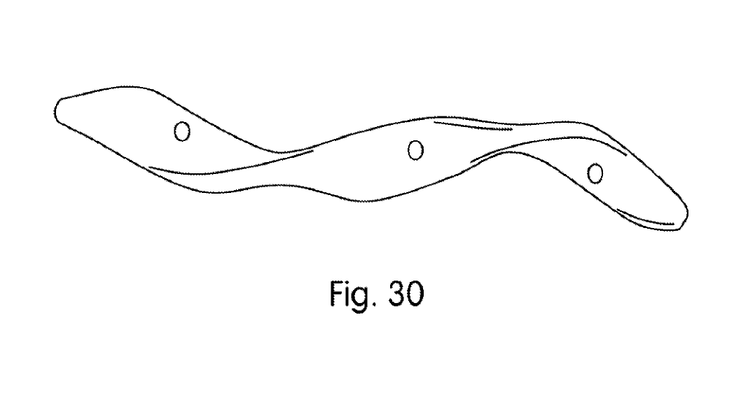

Other designs suited to applications such as vagus nerve stimulation (which may be applied to peripheral nerves in general) may also be adopted and accommodated. A design shown in FIG. 30 that consists of a flexible helix containing exposed micro-transponders on the inner surface, arranged in a manner such that all coils lay parallel to the overlying skin. The array of micro-transponders may have linked electrodes so that they function as a single stimulator, to maximize stimulation around the entire periphery of the nerve. Sizes of micro-transponders can be formed square form-factors of sizes (microns) such as 500×500; 1000×1000; 2000×2000, in rectangular form-factors of sizes (microns) such as 200×500; 250×750; 250×1000.

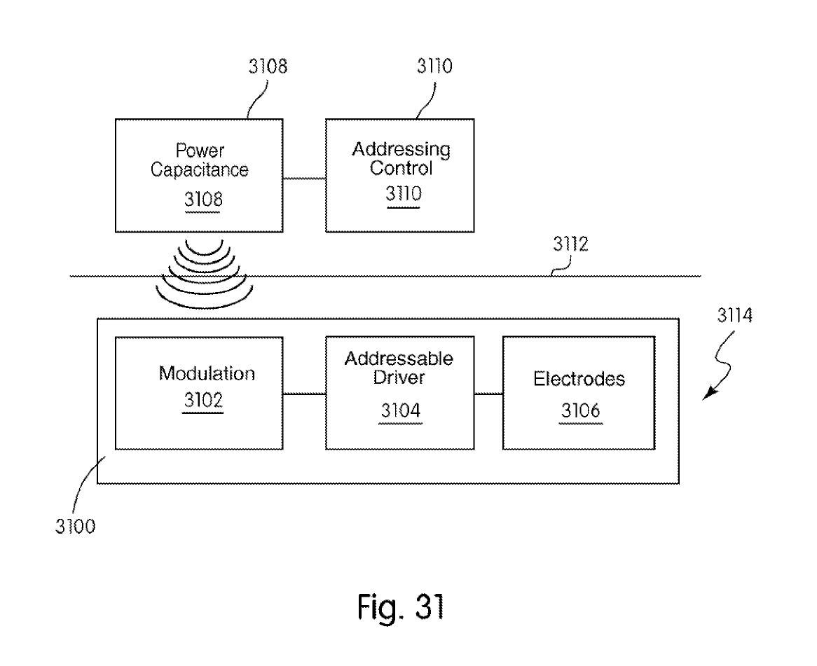

With reference to FIG. 31, a block diagram depicts an individually addressable wireless micro-transponder 3100, in accordance with an embodiment. The individually addressable wireless micro-transponder 3100 may typically include a resonant receiver 3102. The resonant receiver 3102 may be an inductance-capacitance (LC) circuit such as a tank circuit. The resonant receiver 3102 may be connected to an addressable driver 3104. The addressable driver 3104 may receive power, instructions and/or address information from the resonant receiver 3102. The addressable driver 3104 may receive instructions and/or address information from an external source other the resonant receiver 3102. In accordance with the address information received by the addressable driver 3104, the addressable driver 3104 may deliver an electrical current through the electrodes 3106. The passage of electrical current between the electrodes 3106 stimulates the tissue 3114 proximate to the electrodes 3106.

In accordance with an embodiment, the individually addressable wireless micro-transponder 3100 is embedded in human tissue 3114 beneath a layer of skin 3112. A resonant power source 3108 may be tuned to resonate electromagnetic energy at a frequency that generates power in the resonant receiver 3102 of the individually addressable wireless micro-transponder 3100. An addressing control module 3110 may be communicatively connected to the resonant power source 3108 and may provide addressed instructions to the resonant power source 3108 for relay to the resonant receiver 3102. Addressing control 3110 may communicate directly with the addressable driver.

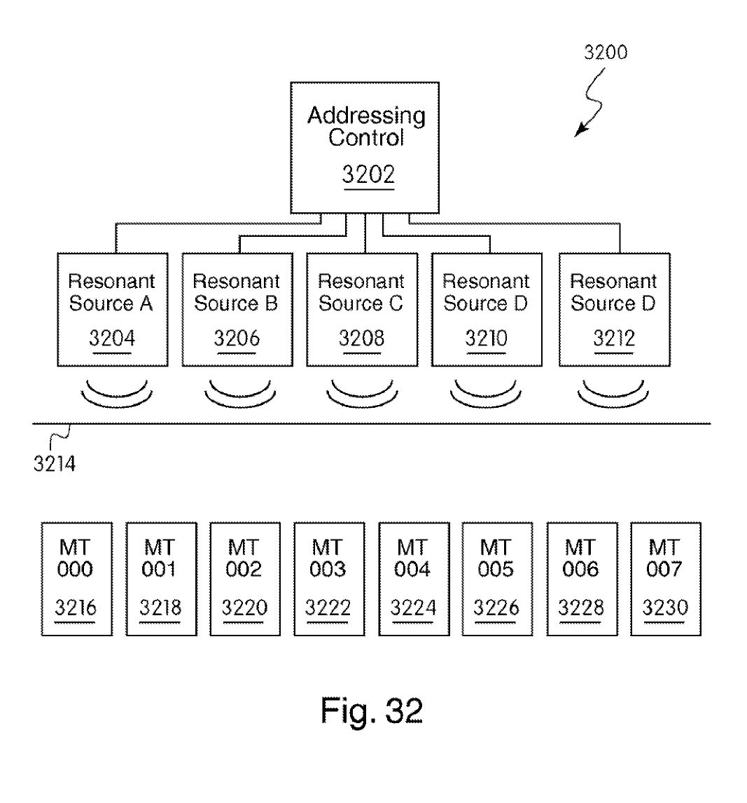

With reference to FIG. 32, a block diagram depicts an addressable wireless micro-transponder system 3200 in accordance with an embodiment. An addressing control module 3202 determines instructions for each of the implanted micro-transponders 3216, 3218, 3220, 3222, 3224, 3226, 3228 and 3230. The instructions in conjunction with the appropriate micro-transponder addresses are communicated to one or several resonant sources 3204, 3206, 3208, 3210 and 3212 in proximity to the addressed micro-transponders 3216, 3218, 3220, 3222, 3224, 3226, 3228 and 3230. For example, the addressing control module 3202 determines to send a stimulation pulse from micro-transponder 3222, having an address=003. The addressing control module 3202 may send an instruction for resonant source C 3208 to provide a signal including the address=003. Although micro-transponders 3220 and 3224 may be sufficiently proximate to the activated resonant source C 3208, only the micro-transponder 3222 having an address=3 will generate the stimulation pulse.

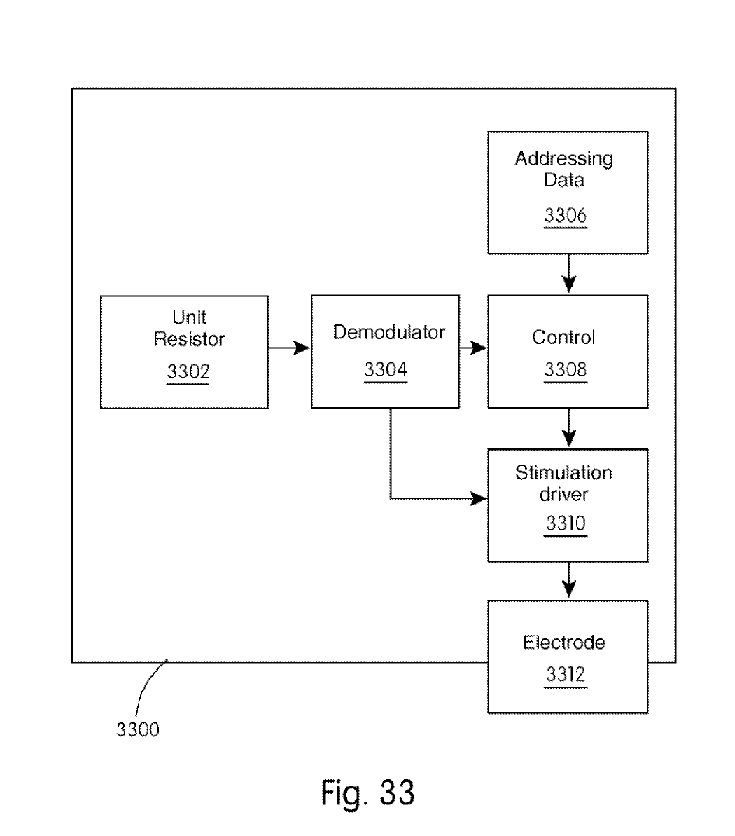

With reference to FIG. 33, a block diagram depicts an addressable micro-transponder 3300. A unit resonator 3302 receives resonated energy output to a demodulator 3304. The demodulator 3304 discriminates data content output to a control circuit 3308. The control circuit 3308 uses addressing data 3306 to filter stimulation instructions output to a stimulation driver 3310. The stimulation driver 3310 outputs a stimulation pulse to an electrode 3312.

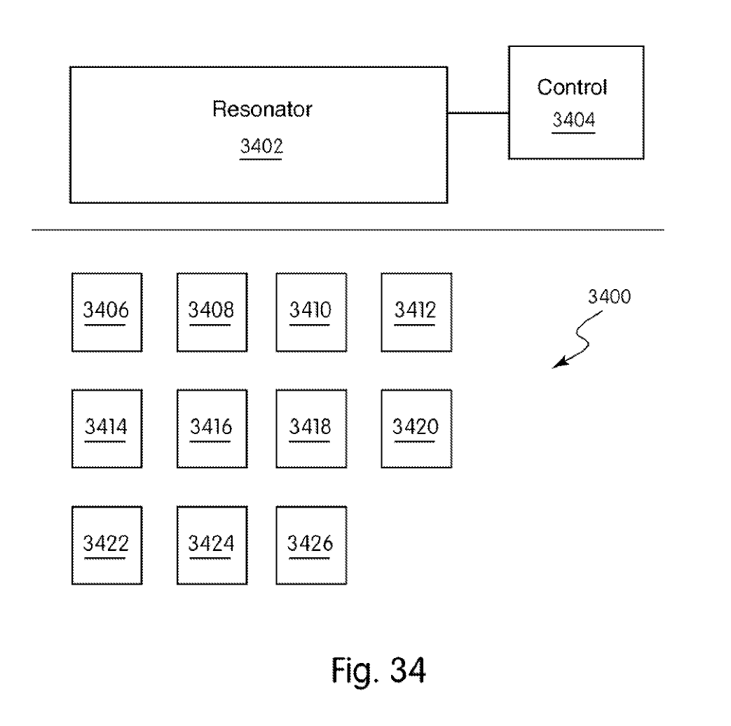

With reference to FIG. 34, a block diagram depicts an addressable micro-transponder system 3400. A resonator 3402 transmits resonant energy in accordance to instructions provided by a control 3404. The micro-transponders 3406, 3408, 3410, 3412, 3416, 3418, 3420, 3422, 3424 and 3426 may be arranged in addressable groups. For example, micro-transponders 3406, 3408, 3410 and 3412 may form a first group, addressable by a group address. Micro-transponders 3414, 3416, 3418 and 3420 may form a second group addressable by a second group address. Micro-transponders 3422, 3424 and 3426 my form a third group.

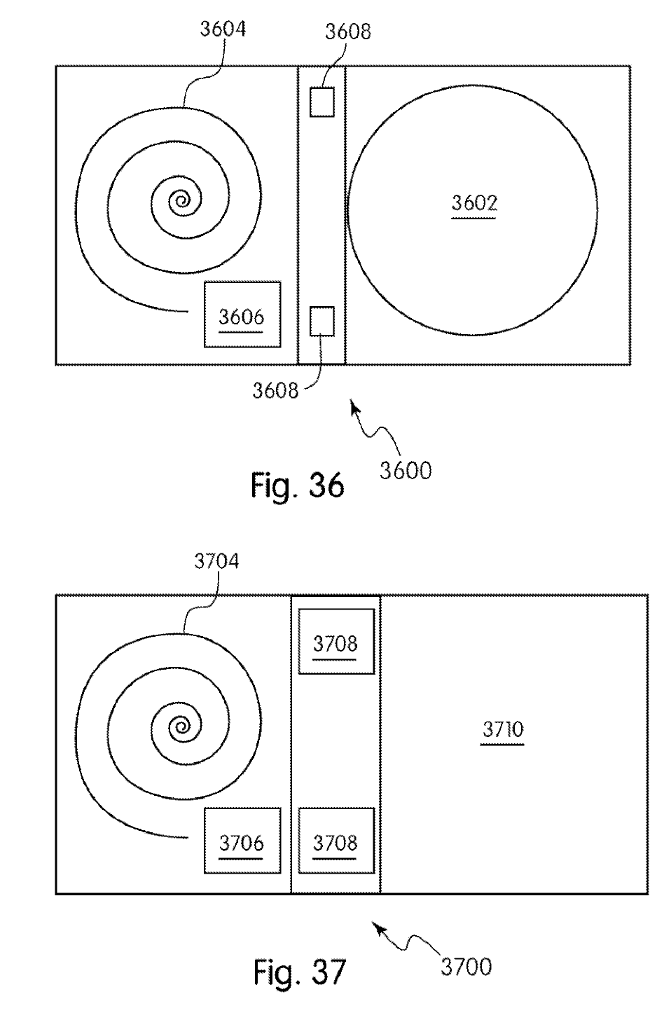

With reference to FIG. 36, a wireless micro-implant platform 3600 is shown. The platform 3600 holds surface electrodes 3602 at one end of the platform 3600 and typically on both the top and the bottom side. An LC resonant circuit is formed with a spiral microcoil 3604 and a capacitance 3606. Rectifier diodes 3608 are positioned between the resonant circuit and the electrodes 3602. The surface electrodes 3602 may be used for neural stimulation, or any other suitable use.

With reference to FIG. 37, a wireless micro-implant platform 3700 is shown. The platform includes an ASIC socket 3710 at one end of platform 3700. An LC resonant circuit is formed with a flat spiral microcoil 3704 and a capacitance 3706. Rectifier diodes 3708 may be positioned between the resonant circuit and the electrodes 3702.

### Modifications and Variations

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

Although described to provide numerous features and advantages, the present embodiments could include minimal transponder circuits, for example, as a coil connected to a capacitance and a rectifier.

A voltage booster may be inserted immediately after the rectifier element 318 to boost the supply voltage available for stimulation and operation of integrated electronics beyond the limits of what might be generated by a miniaturized LC resonant tank circuit. The voltage booster may enable electro-stimulation and other micro-transponder operations using the smallest possible LC components, which may generate too little voltage, for example, less than 0.5 volts.

Examples of high efficiency voltage boosters include charge pumps and switching boosters using low-threshold Schottky diodes. However, it should be understood that any type of conventional high efficiency voltage booster may be utilized in this capacity as long as it can generate the voltage required by the particular application that the micro-transponder is applied to.

Micro-transponders may not be physically linked while inside the cannula and stored in low temperature, such as around 40 C; the physically linked array may be formed after the injection by a biocompatible get like material, such as Matrigel™ (a product of BD Biosciences, Inc.), that solidifies when exposed to higher temperature, such as body temperature, and the space between each micro-transponder may be adjusted by the pushing speed.

The shape of cannula, width, thickness and length vary for different purposes and clinic uses, for example, for deep tissue injection, the cannula may be made of strong material of sharper edge with a long extended body.

For example, in one embodiment, rather than an elongated strip, the linked micro-transponders can be linked both longitudinally and latitudinally to form a geometric shape. The shapes can include squares, hexagons, rectangles, ovals, and circles.

The array can also be formed on a single substrate, with a chain or group of arrays constructed contemporaneously to form a single integrated structure. It may also be possible to construct linked arrays using a monofilament line as a string of arrays.

One such specific variation is dispensing with the subdermal/outer transfer coil to use a three coil power transmission arrangement. Power from the external coil would transmit to the subcutaneous/inner transfer coil which would power the micro-transponder micro-coil. The interface between the two transfer coils can comprise radio frequency, low frequency, or direct current power. The wired connection between the two transfer coils can typically be coaxial or balanced line connection. The external coil and the subdermal/outer transfer coil can comprise paralleled coils at the skin surface. There can further be multiple internal drivers to power the micro-transponders. The configuration can make use of spatial resolution. Finally, the described embodiment is a single power transfer through one internal tissue boundary, while the invention also extends to a double through two internal boundaries or potentially more.

It is also possible to vary the power source in the invention. The connection between the subdermal (or outer transfer) coil and subcutaneous (or inner transfer) coil does not necessarily have to be a connection at the resonant RF frequency. In alternative embodiments, it is contemplated that this power-transfer connection can be DC, or can be AC at a lower frequency than RF, or a non-resonating AC frequency of the micro-transponder micro-coils. If the connection is DC, a power conversion stage would be included in the outer transfer coil circuitry, to convert the received RF power to DC. This can be quite similar to the AC-DC conversion which is normally used to charge up the storage capacitor for stimulation pulses. In this case, the inner transfer coil would need to contain or be combined with an oscillator of some sort, to generate an AC signal (for wireless coupling) from the received DC power. Similar adaptation is used if the connecting link operates at a lower AC frequency on non-resonating AC frequency, with a converter circuit generating an AC signal compatible with the micro-transponder micro-coils and power circuits.

According to various embodiments, there is provided a method of providing electrical stimulation to tissue comprising: implanting one or more battery-free micro-transponders, having spiral antennas integrated therewith, into tissue; wirelessly providing energy to said plurality of micro-transponders; and stimulating said tissue with said energy.

According to various embodiments, there is provided a micro-transponder system comprising an external interface unit; a battery-free internal transponder wirelessly receiving energy from said external interface unit; and a stimulation driver powered from said internal transponder.

According to various embodiments, there is provided a micro-transponder comprising an antenna; an energy storage circuit connected to store energy received at said antenna; and a stimulator circuit connected to apply pulses to biocompatible electrodes.

According to various embodiments, there is provided a neural stimulation device comprising biocompatible electrodes providing stimulation energy to peripheral nerve tissue; and an inductively coupled stimulation energy source connected to said biocompatible electrodes.

According to various embodiments, there is provided a method for providing neural stimulation comprising receiving energy with an implanted tank circuit; and providing intermittent stimulation pulses from said implanted tank circuit to biocompatible electrodes in contact with neural tissue.

According to various embodiments, there is provided a method of providing neural stimulation comprising receiving externally generated energy by inductive coupling with a flat spiral coil to generate stimulation energy; and providing said stimulation energy to peripheral nerve tissue.

According to various embodiments, there is provided a method and system for providing electrical stimulation to tissue includes implanting one or more batter-free micro-transponders having spiral antennas into tissue. Energy is provided wirelessly to the plurality of micro-transponders. Tissue is stimulated using the energy.

The following applications may contain additional information and alternative modifications: Attorney Docket No. MTSP-29P, Ser. No. 61/088,099 filed Aug. 12, 2008 and entitled “In Vivo Tests of Switched-Capacitor Neural Stimulation for Use in Minimally-Invasive Wireless Implants; Attorney Docket No. MTSP-30P, Ser. No. 61/088,774 filed Aug. 15, 2008 and entitled “Micro-Coils to Remotely Power Minimally Invasive Microtransponders in Deep Subcutaneous Applications”; Attorney Docket No. MTSP-31P, Ser. No. 61/079,905 filed Jul. 8, 2008 and entitled “Microtransponders with Identified Reply for Subcutaneous Applications”; Attorney Docket No. MTSP-33P, Ser. No. 61/089,179 filed Aug. 15, 2008 and entitled “Addressable Micro-Transponders for Subcutaneous Applications”; Attorney Docket No. MTSP-34P, Ser. No. 61/078,954 filed Jul. 8, 2008 and entitled “Neuroplastivity Enhancement”; Attorney Docket No. MTSP-35P Ser. No. 61/077,648 filed Jul. 2, 2008 and entitled “Treatment of Tinnitus with Vegus Nerve Stimulation”; Attorney Docket No. MTSP-36P Ser. No. 61/079,004 filed Jul. 8, 2008 and entitled “Microtransponder Array with Biocompatible Scaffold”; Attorney Docket No. MTSP-38P Ser. No. 61/083,290 filed Jul. 24, 2008 and entitled “Minimally Invasive Microtransponders for Subcutaneous Applications” Attorney Docket No. MTSP-39P Ser. No. 61/086,116 filed Aug. 4, 2008 and entitled “Tintinnitus Treatment Methods and Apparatus”; Attorney Docket No. MTSP-40P, Ser. No. 61/086,309 filed Aug. 5, 2008 and entitled “Wireless Neurostimulators for Refractory Chronic Pain”; Attorney Docket No. MTSP-41P, Ser. No. 61/086,314 filed Aug. 5, 2008 and entitled “Use of Wireless Microstimulators for Orofacial Pain”; Attorney Docket No. MTSP-42P, Ser. No. 61/090,408 filed Aug. 20, 2008 and entitled “Update: In Vivo Tests of Switched-Capacitor Neural Stimulation for Use in Minimally-Invasive Wireless Implants”; Attorney Docket No. MTSP-43P, Ser. No. 61/091,908 filed Aug. 26, 2008 and entitled “Update: Minimally Invasive Microtransponders for Subcutaneous Applications”; Attorney Docket No. MTSP-44P, Ser. No. 61/094,086 filed Sep. 4, 2008 and entitled “Microtransponder MicroStim System and Method”; Attorney Docket No. MTSP-30, Ser. No. 12/323,904, filed Nov. 26, 2008 and entitled “Transfer Coil Architecture”; Attorney Docket No. MTSP-31, Ser. No. 12/323,934, filed Nov. 26, 2008 and entitled “Implantable Driver with Charge Balancing”; Attorney Docket No. MTSP-32, Ser. No. 12/323,952, filed Nov. 26, 2008 and entitled “A Biodelivery system for Microtransponder Array”; Attorney Docket No. MTSP-46, Ser. No. 12/323,969, filed Nov. 26, 2008 and entitled “Implanted Driver with Resistive Charge Balancing”; Attorney Docket No. MTSP-47, Ser. No. 12/324,000, filed Nov. 26, 2008 and entitled “Array of Joined Microtransponders for Implantation”; and Attorney Docket No. MTSP-48, Ser. No. 12/324,044, filed Nov. 26, 2008 and entitled “Implantable Transponder Pulse Stimulation Systems and Methods” and all of which are incorporated by reference herein.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rl, and an upper limit, Ru, is disclosed, any number falling within the range is specifically disclosed. In particular, the following numbers within the range are specifically disclosed: R=Rl+k*(Ru−Rl), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. The use of the term about means ±10% of the subsequent number, unless otherwise stated. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present disclosure. The discussion of a reference in the disclosure is not an admission that it is prior art, especially any reference that has a publication date after the priority date of this application. The disclosure of all patents, patent applications, and publications cited in the disclosure are hereby incorporated by reference, to the extent that they provide exemplary, procedural, or other details supplementary to the disclosure.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

What is claimed is:

1. A linear implantable device array comprising:
a plurality of implantable devices, wherein each of the plurality of implantable devices comprises:
an antenna;
an energy storage circuit connected to the antenna;
a stimulator circuit connected to the energy storage circuit and applying pulses to a plurality of electrodes; and
a connecting material, wherein the connecting material connects each of the implantable devices to form a physically-connected array of implantable devices,
wherein the implantable devices are collectively housed in a cannula, and
wherein the cannula is configured to implant the physically-connected array of implantable devices into a tissue at a single injection point.

2. The array of claim 1, wherein the antenna is an inductive antenna.

3. The array of claim 1, wherein the energy storage circuit receives energy from the antenna.

4. The array of claim 1, wherein the stimulator circuit receives instructions from the antenna.

5. The array of claim 4, wherein the stimulator circuit receives stimulation timing instructions.

6. The array of claim 4, wherein the stimulator circuit receives stimulation parameter instructions.

7. The array of claim 1, wherein the plurality of implantable devices includes three implantable devices.

8. The array of claim 1, wherein the antenna is a flat spiral coil.

9. The array of claim 1, wherein the energy storage circuit is a capacitor.

10. The array of claim 1, wherein the connecting material is a polymer.

11. A method of providing electrical stimulation to tissue comprising:
implanting into tissue an array of independent implantable devices, each independent implantable device including an antenna and electrodes, wherein each of the independent implantable devices is physically connected to at least another one of the independent implantable devices in the array with a connecting material, and wherein the physically-connected array of independent implantable devices is housed within a cannula prior to being implanted into the tissue;
providing energy through skin to each of the antennas at the same time using a single control device;
communicating instructions from the single control device to each of the independent implantable devices using the energy;
receiving response replies from each of the independent implantable devices by the single control device; and
stimulating the tissue using one or more of the independent implantable devices in accordance with the instructions.

12. The method of claim 11, wherein the energy is inductive energy.

13. The method of claim 11, wherein the instructions are communicated serially to each of the independent implantable devices.

14. The method of claim 11, wherein the instructions include an address identifying one of the independent implantable devices.

15. The method of claim 11, wherein the instructions include stimulation timing instructions.

16. The method of claim 11, wherein the instructions include stimulation parameter instructions.

17. The method of claim 11, wherein implanting into tissue the physically-connected array of independent implantable devices comprises inserting the cannula into the tissue at a single injection point.

18. The method of claim 17, wherein implanting into tissue the physically-connected array of independent implantable devices comprises retracting the cannula from the tissue while simultaneously expelling the physically-connected array of independent implantable devices from the cannula into the tissue at the single injection point.

19. The method of claim 11, wherein the single control devices provides energy inductively.

20. The method of claim 11, wherein the single control device provides instructions using radio frequency communication.
